SEQUENCE LISTING (5 sequences)

SEQ ID NO: 1 - protein, 375 aa, Homo sapiens
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 2 - DNA, 1852 bp, Homo sapiens
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaaaaaaaaaaaaaaaaaaaaaaaaaaaaaaaaaaaaaaaaa

SEQ ID NO: 3 - protein, 44 aa, Homo sapiens
MSDKPDMAEIEKFDKSKLKKTETQEKNPLPSKETIEQEKQAGES

SEQ ID NO: 4 - DNA, 657 bp, Homo sapiens
gacaactcggtggtggccactgcgcagaccagacttcgctcgtactcgtgcgcctcgcttcgcttttcctccgcaaccatgtctgacaaacccgatatggctgagatcgagaaattcgataagtcgaaactgaagaagacagagacgcaagagaaaaatccactgccttccaaagaaacgattgaacaggagaagcaagcaggcgaatcgtaatgaggcgtgcgccgccaatatgcactgtacattccacaagcattgccttcttattttacttcttttagctgtttaactttgtaagatgcaaagaggttggatcaagtttaaatgactgtgctgcccctttcacatcaaagaactactgacaacgaaggccgcgcctgcctttcccatctgtctatctatctggctggcagggaaggaaagaacttgcatgttggtgaaggaagaagtggggtggaagaagtggggtgggacgacagtgaaatctagagtaaaaccaagctggcccaaggtgtcctgcaggctgtaatgcagtttaatcagagtgccatttttttttttgttcaaatgattttaattattggaatgcacaatttttttaatatgcaaataaaaagtttaaaaacttaaaaaaaaaaaaaaaaaaaaaaaaaaaaa

SEQ ID NO: 5 - protein, 44 aa, Homo sapiens; identical to SEQ ID NO: 3

(19) United States
(12) Patent Application Publication
(10) Pub. No.: US 2017/0307609 A1
(43) Pub. Date: Oct. 26, 2017

(54) Methods for Treating Sepsis and Biomarkers Related Thereto

(71) Applicant: Henry Ford Health System, Detroit, MI (US)

(72) Inventors: Justin Brett Belsky, Hamden, CT (US); Daniel Claude Morris, Grosse Pointe Park, MI (US); Emanuel Rivers, Detroit, MI (US)

(21) Appl. No.: 15/516,193

(22) PCT Filed: Oct. 1, 2015
(86) PCT No.: PCT/US15/53505

§ 371 Date: Mar. 31, 2017

Related U.S. Application Data
(60) Provisional application No. 62/058,340, filed on Oct. 1, 2014.

(51) Int. Cl. G01N 33/569; G01N 33/68; G01N 33/53; C12Q 1/68

(52) U.S. Cl. CPC G01N 33/56911; G01N 33/53; G01N 33/6893; G01N 2333/90; C12Q 1/68; G01N 2800/26; G01N 2800/52; G01N 2333/47

(57) ABSTRACT

The present invention provides a method of treating, preventing, diagnosing and prognosing sepsis, and septic shock, and subjects likely to progress to sepsis and subjects in septic shock using biomarkers that can be used to stratify treatment procedures in response to the diagnosis.

### REFERENCE TO CROSS RELATED APPLICATIONS

This application claims the benefit of U.S. Provisional Application No. 62/058,340 filed on Oct. 1, 2014, the disclosure of which is incorporated by reference herein in its entirety.

### SEQUENCE LISTING

This application incorporates by reference in its entirety the Sequence Listing entitled “25824-375899_Sequence Listing_ST25.txt” (7.72 kilobytes), which was created on Oct. 1, 2015 and filed electronically herewith.

### FIELD

The present invention generally relates to methods for treating sepsis and biomarkers useful in the identification of patients that are progressing to sepsis.

### BACKGROUND

The statements in this section merely provide background information related to the present disclosure and may not constitute prior art. Septic shock requires prompt treatment since the patient's condition often deteriorates rapidly. Symptoms of septic shock include fever, hypothermia, falling blood pressure, rapid breathing, rapid heartbeat, skin lesions and leakage of plasma proteins into the tissues, metabolic acidosis and elevated plasma lactate. Septic shock is particularly characterized by maldistribution of blood flow and disturbances in tissue oxygen in various organs of the body. Distribution of blood flow may become heterogeneous with subsequent under- and over perfusion of various tissues. These disturbances have been noted both at the macro-as well as at the microcirculatory level. Septic shock is the leading cause of morbidity and mortality in the intensive care units. Despite increased knowledge about the pathophysiology underlying the clinical symptoms mortality remains high and has not decreased substantially over the last decades.

There are several causes of septic shock including bacterial, fungal and viral infections as well as noninvasive stimuli such as multiple trauma, severe burns, organ transplantations and pancreatitis. The fatal outcome of septic shock has recently been linked to the systemic release of substantial amounts of various cytokines in the body.

Septic patient usually die as a result of poor tissue perfusion and injury followed by multiple organ failure. At least 7 million patients per year enter into the early stages of the sepsis pathology, a medical condition named systemic inflammatory response syndrome (SIRS), which will lead to more than 250.000 deaths per year in the USA and more than 750,000 worldwide. It is predicted that larger numbers of persons will develop SIRS as the population ages. Sepsis develops from a variety of bacterial and fungal sources stemming from the patient's inability to fight infection, and is commonly acquired while recovering from severe injuries and surgery in hospitals.

Several published studies describe the need for the early diagnosis of the enormous sepsis pathology to provide early patient treatment and to rearrange the costs of treatment. There is also a need for supplemental tests for the sepsis pathology. Namely, supplemental tests are needed to provide data: (1) To differentiate between patients who are suspected of becoming septic and who will not develop sepsis and those patients who are suspected of becoming septic and who will become septic, severely septic or suffer from septic shock; (2) To determine the susceptibility of an individual patient to becoming septic; (3) To place an individual patient's current status as “very early”, “early”, or “mid-stage” in an episode of sepsis; and (4) Regarding the probability that an individual patient's condition is expected to deteriorate or to improve.

Current laboratory culture procedures for diagnosing sepsis suffer from a number of problems including lack of reliable methods to provide culture results within 48 hours. A second major deficiency is the qualitative accuracy of the test results, including false negatives, wherein blood culture only yields positive results (i.e. sepsis is present) in approximately 28% of patients who become septic, Thus, over 70% of the patients do not yield positive blood cultures.

Most often, sepsis starts with a bacterial or fungal infection, but the pathology results from an individual patient's hyperinflammatory response to bacterial cellular antigens which are produced when the body attempts to fight off the infection where such microorganisms are killed. The patient's response to these available cell wall and other cellular antigens initiates the cascade of events leading to a “cytokine storm”.

Many pharmacological human clinical trials have been aimed against hindering the inflammatory pathway in sepsis, but all have failed. Some notable drugs include Drotrecogin (a recombinant form of human activated protein C), anti-TNFα, and anti-IL-1 therapy. Therapeutic drug trials in pediatric septic shock have been universal failures to date. The most recent and notable example is that of activated protein C (APC). APC was recently approved by the FDA as the only drug specifically labeled for septic shock in adults. A phase III trial of APC in children was recently terminated at interim analysis secondary to lack of efficacy and a trend toward increased complications. There is a well-founded perception that a primary reason why the pediatric APC trial failed was because many of the enrolled patients were destined to do well with standard care (i.e. they were not “sick” enough). Thus, when patients are enrolled into a drug trial having significant risks (hemorrhage), and they have a high likelihood of doing well with standard care, the risk to benefit ratio is negatively impacted for the overall patient cohort. Thus, there is a need for more effective stratification of sepsis patients at the time of enrollment.

The difficulty in early diagnosis of sepsis is reflected by the high morbidity and mortality associated with the disease. Reported figures provided from the Centers of Disease Control (CDC) in 2010, sepsis is the tenth leading cause of death in the United States for women, children aged 1-9, adults aged 45-85, and is especially prevalent among hospitalized patients in non-coronary intensive care units (ICUs). The overall rate of mortality is as high as 35%, with an estimated 750,000 cases per year occurring in the United States alone. The annual cost to treat sepsis in the United States alone is on the order of billions of dollars. While research into the causes and treatments for sepsis is an ongoing concern, multiple trials have focused on immune modulation strategies in adults with septic shock. Despite strong preclinical data, as well as strong phase I and II data, the majority of these strategies have failed when subjected to large scale, randomized placebo-controlled trials. Consequently, the majority of these strategies have not been effectively tested in the pediatric population. Current care for pediatric septic shock remains fundamentally based on antibiotics and supportive care.

Many biomarkers have been elucidated, but none appear to be specific to sepsis. TNF-A, IL-1B, and IL-6, all pro-inflammatory cytokines, are elevated in sepsis. Other inflammatory markers, classified as chemokines secondary to their ability to attract inflammatory cells, such as IL-8 and monocyte chemoattractant protein, are also associated with sepsis.

Actin is an abundant protein present in most eukaryotic cells and participates in numerous protein-protein interactions influencing, cell morphology, muscle contraction (Rayment 1993), and cell motility (Dominguez 2011). It is a 42 kDa. globular protein (Elzinga 1973) that cycles between a monomeric (G-actin) and filamentous (F-actin) state. The intracellular pool of monomeric G-actin is complexed to and regulated by numerous actin binding proteins (ABPs) which regulate the conversion of G- to F-actin (Xue 2013).

Thymosin Beta 4 (TB4) is expressed in almost all eukaryotic cells. Its main intracellular activity is to bind G-actin into a 1:1 complex, rendering G-actin resistant to polymerization into its filamentous F-actin form. TB4 is important in maintaining a large intracellular volume of monomeric actin that is readily available for use if needed (Mannherz 2009). TB4 has other activities including preventing apoptosis by decreasing cytochrome c release from mitochondria, increasing bcl-2 expression, and decreasing caspase activation (Sosne 2004). Additionally, mice exposed to endotoxin-induced septic shock had decreased mortality when pre-treated with exogenous TB4 (Badamchian 2003), suggesting a role for TB4 and the inhibition of G-actin to F-actin in the pathogenesis of sepsis.

For at least the reasons provided above, there is a need to provide biological markers that are prognostically useful in identifying patients that are likely to progress to septic shock if left untreated, or to accurately diagnose a patient suspected of having sepsis, or septic shock.

### SUMMARY

The present technology provides methods for diagnosing and prognosing the presence of sepsis and septic shock in a subject.

In another aspect the present technology provides a method for diagnosing or prognosing sepsis in a subject comprising the steps: (a) providing a biological sample from the subject suspected of having sepsis or a subject likely to develop sepsis or having septic shock; (b) determining the expression level of G-actin and F-actin in the biological sample; and (c) correlating the ratio of F-actin expression and G-actin expression (F-actin/G-actin) in the biological sample to a known standard.

In another aspect the present technology provides a method for treating or preventing septic shock in a non-infectious or infectious SIRS subject. In some examples, the method comprises: (a) obtaining a blood sample from the non-infectious SIRS subject or the infectious SIRS subject; (b) determining the amount of F-actin in the non-infectious SIRS subject or the infectious SIRS subject's blood sample; (c) determining that the non-infectious SIRS subject or the infectious SIRS subject is in severe sepsis or septic shock if the non-infectious SIRS subject or the infectious SIRS subject's F-actin level is about 3 ng/mL or greater; and (d) administering an effective treatment to treat or prevent septic shock in the non-infectious SIRS subject or the infectious SIRS subject having an F-actin level of about 3 ng/mL or greater.

Further areas of applicability will become apparent from the description provided herein. It should be understood that the description and specific examples are intended for purposes of illustration only and are not intended to limit the scope of the present disclosure.

### DETAILED DESCRIPTION

The following description is merely exemplary in nature and is not intended to limit the present disclosure, application, or uses.

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein. All references cited in the “Description” section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made, or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

Although the open-ended term “comprising,” as a synonym of terms such as including, containing, or having, is use herein to describe and claim the present invention, the invention, or embodiments thereof; may alternatively be described using more limiting terms such as “consisting of” or “consisting essentially of” the recited ingredients.

The practice of the present invention will employ, unless otherwise indicated, conventional techniques of molecular biology (including recombinant techniques), microbiology, immunology, cell biology and biochemistry, which are within the skill of the art.

“Systemic inflammatory response syndrome,” or “SIRS,” refers to a clinical response to a variety of severe clinical insults, as manifested by two or more of the following conditions within a 24-hour period: a body temperature greater than 38° C. (100.4° F.) or less than 36° C. (96.8° F.); a heart rate (HR) greater than 90 beats/minute; a respiratory rate (RR) greater than 20 breaths/minute, or a P_{CO2} less than 32 mmHg, or requiring mechanical ventilation; and white blood cell count (WBC) either greater than 12.0×10⁹/L or less than 4.0×10⁹/L. A subject with SIRS has a clinical presentation that is classified as SIRS, as defined above, but is not clinically deemed to be septic. Such subjects include, for example, those in an ICU and those who have otherwise suffered from a physiological trauma, such as a burn, surgery or other insult. A hallmark of SIRS is the creation of a proinflammatory state that can be marked by tachycardia, tachypnea or hyperpnea, hypotension, hypoperfusion, oliguria, leukocytosis or leukopenia, pyrexia or hypothermia and the need for volume infusion. SIRS characteristically does not include a documented source of infection (e.g., bacteremia).

These symptoms of SIRS represent a consensus definition of SIRS that can be modified or supplanted by other definitions in the future. The present definition is used to clarify current clinical practice and does not represent a critical aspect of the invention (see, e.g., American College of Chest Physicians/Society of Critical Care Medicine Consensus Conference: Definitions for Sepsis and Organ Failure and Guidelines for the Use of Innovative Therapies in Sepsis, 1992, Crit. Care. Med. 20, 864-874, the entire contents of which are herein incorporated by reference).

A “biological sample” encompasses any sample obtained from a living system or subject. The definition encompasses blood, plasma, serum, tissue, and other samples of biological origin that can be collected from a living system, subject or individual. Preferably, biological samples are obtained through sampling by minimally invasive or non-invasive approaches (e.g., urine collection, stool collection, blood drawing, needle aspiration, and other procedures involving minimal risk, discomfort or effort). Biological samples can be gaseous (e.g., exhaled breath). Biological samples are often liquid (sometimes referred to as a “biological fluid”). Liquid biological samples include, but are not limited to, urine, blood, plasma, serum, interstitial fluid, edema fluid, saliva, lacrimal fluid, inflammatory exudates, synovial fluid, abscess, empyema or other infected fluid, cerebrospinal fluid, sweat, pulmonary secretions (sputum), seminal fluid, feces, bile, intestinal secretions, and others. Biological samples include samples that have been manipulated in any way after their procurement, such as by treatment with reagents, solubilization, or enrichment for certain components, such as proteins or polynucleotides. The term “biological sample” also encompasses a clinical sample such as serum, plasma, other biological fluid, or tissue samples, and also includes cells in culture, cell supernatants and cell lysates.

As used herein, the terms “pharmaceutically active agent,” “medicament”, “drug”, “bioactive agent”, “therapeutic agent”, and “active agent” may be used interchangeably and refer to a substance, such as a small molecule chemical compound or complex, or natural polymer, such as antibodies, or nucleic acids, that have a measurable beneficial physiological effect on the body, such as a therapeutic effect in treatment of a disease or disorder, when administered in an effective amount. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, prodrugs, active metabolites, isomers, fragments, analogs, solvates hydrates, radioisotopes, etc.

The present application provides stratified levels of treatment or intervention for patients diagnosed or having a prognosis of SIRS, sepsis, and septic shock. As used herein, the term: “Higher risk” or “aggressive” therapy will be understood by one of ordinary skill in the art and includes, for example, plasmapheresis, high dose ultrafiltration, extracorporeal membrane oxygenation, selective cytopheresis, selective antigen removal, and/or continuous renal replacement therapy. Such therapies are also intended to include newly developed therapies (e.g., active agents or invasive procedures) considered to be higher risk therapies, and active agents that are considered higher risk by one of skill in the art. Specific support for the possible treatments encompassed by “Higher risk” or “aggressive” therapy, see Carcilllo, et al, “Clinical practice parameters for hemodynamic support of pediatric and neonatal patients in septic shock,” Crit. Care Med. 2002 June; 30(6):1365-78, and Annane D. et al, “Septic Shock,” Lancet, 2005 Jan. 1-7; 365(9453):63-68, the disclosures of which are incorporated herein by reference in their entireties.

The phrase “standard care” with respect to septic shock is known to one of ordinary skill in the art and generally includes antibiotics and organ support.

A “subject” as exemplified herein, is a vertebrate, preferably a mammal, preferably a human. In some embodiments, subjects are experimental laboratory animals such as mice, rats, rabbits and other animals. In various embodiments, a subject also includes companion animals such as dogs, cats, and horses. The terms “subject” and “patient” are used interchangeably herein.

The term “therapeutic” treatment is well known in the medical arts and refers to administration to the subject of one or more pharmaceutically active agents, medicaments, drugs, bioactive agents, therapeutic agents, or active agents. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The phrase “therapeutic effect” is well known in the medical arts, and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the individual and disease condition being treated, the weight and age of the individual, the severity of the disease condition, the manner of administration and the like, which can readily be, determined by one of ordinary skill in the art.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Sepsis” refers to a systemic host response to infection with SIRS plus a documented infection (e.g., a subsequent laboratory confirmation of a clinically significant infection such as a positive culture for an organism). Thus, sepsis refers to the systemic inflammatory response to a documented infection (see, e.g., American College of Chest Physicians Society of Critical Care Medicine, Chest, 1997, 101:1644-1655, the entire contents of which are herein incorporated by reference).

As used herein, the term “septic shock” refers to a clinically well-defined condition known in the art, and exists in a subject exhibiting the symptoms of fever, hypothermia, falling blood pressure, rapid breathing, rapid heartbeat, skin lesions and leakage of plasma proteins into the tissues, metabolic acidosis and elevated plasma lactate. Septic shock is particularly characterized by maldistribution of blood flow and disturbances in tissue oxygen in various organs of the body. Distribution of blood flow may become heterogeneous with subsequent under- and overperfusion of various tissues. These disturbances have been noted both at the macro-as well as at the microcirculatory level.

The phrase “standard care” with respect to septic shock is known to one of ordinary skill in the art and generally includes antibiotics and organ support.

The term “preexisting condition” generally defines a patient or subject population that exhibits symptoms, or is confirmed to have a disease, or disorder that may, in some circumstances, also progress to sepsis, or septic shock as a primary or secondary comorbidity. In some illustrative embodiments, a subject with a preexisting condition, can include a subject diagnosed with an infection, a subject diagnosed with SIRS, a subject suspected of having SIRS, a subject having one or more symptoms of an inflammatory condition, a subject diagnosed with an autoimmune disease, a subject having a surgery performed less than 72 hours, a subject admitted for medical treatment as a result of a trauma, a subject admitted for medical treatment as a result of a burn, a premature neonatal subject, and a subject diagnosed with a cardiovascular disease.

The “onset of sepsis” refers to an early stage of sepsis, e.g., prior to a stage when conventional clinical manifestations are sufficient to support a clinical suspicion of sepsis. Because the methods of the present invention are used to detect those subjects that are likely to progress to septic shock or have septic shock prior to a time that septic shock would be suspected using conventional techniques, the subject's disease status at early sepsis can only be confirmed retrospectively, when the manifestation of sepsis is more clinically obvious. The exact mechanism by which a subject becomes septic is not a critical aspect of the invention. The methods of the present invention can detect the onset of sepsis independent of the origin of the infectious process.

A non-infectious SIRS subject or patient refers to a subject or patient with two or more of the following conditions within a 24-hour period: a body temperature greater than 38° C. (100.4° F.) or less than 36° C. (96.8° F.); a heart rate (HR) greater than 90 beats/minute; a respiratory rate (RR) greater than 20 breaths/minute, or a PCO₂ less than 32 mmHg, or requiring mechanical ventilation; and white blood cell count (WBC) either greater than 12.0×10⁹/L or less than 4.0×10⁹/L and has not been tested with a positive culture, bacteremia or infection.

An infectious SIRS subject or patient refers to a subject or patient with two or more of the following conditions within a 24-hour period: a body temperature greater than 38° C. (100.4° F.) or less than 36° C. (96.8° F.); a heart rate (HR) greater than 90 beats/minute; a respiratory rate (RR) greater than 20 breaths/minute, or a PCO₂ less than 32 mmHg, or requiring mechanical ventilation; and white blood cell count (WBC) either greater than 12.0×10⁹/L or less than 4.0×10⁹/L and has been tested with a positive culture, bactrenia or infection. A non-infectious SIRS subject or patient refers to a subject or patient with at least one of the above conditions, and has a negative culture, or bacteremia or infection.

“Severe sepsis” refers to sepsis associated with organ dysfunction, hypoperfusion abnormalities, or sepsis-induced hypotension. Hypoperfusion abnormalities include, but are not limited to, lactic acidosis, oliguria, or an acute alteration in mental status.

“Septic shock” refers to sepsis-induced hypotension that is not responsive to adequate intravenous fluid challenge and with manifestations of peripheral hypoperfusion.

A “biomarker” is virtually any detectable compound, such as a protein, a peptide, a proteoglycan, a glycoprotein, a lipoprotein, a carbohydrate, a lipid, a nucleic acid (e.g., DNA, such as cDNA or amplified DNA, or RNA, such as mRNA), an organic or inorganic chemical, a natural or synthetic polymer, a small molecule (e.g., a metabolite), or a discriminating molecule or discriminating fragment of any of the foregoing, that is present in or derived from a biological sample. “Derived from” as used in this context refers to a compound that, when detected, is indicative of a particular molecule being present in the biological sample. For example, detection of a particular cDNA can be indicative of the presence of a particular RNA transcript in the biological sample. As another example, detection of or binding to a particular antibody can be indicative of the presence of a particular antigen (e.g., protein) in the biological sample. Here, a discriminating molecule or fragment is a molecule or fragment that, when detected, indicates presence or abundance of an above-identified compound.

A biomarker, for example, G-actin and/or F-actin, and/or TB4 can be isolated from the biological sample, for example a blood sample, directly measured in the biological sample, or detected in or determined to be in the biological sample. A biomarker, G-actin and/or F-actin, and/or TB4 can, for example be functional, partially functional, or non-functional. In one embodiment of the present invention, G-actin and/or F-actin and/or TB4 are isolated and used, for example, to raise a specifically-binding antibody that can facilitate detection of G-actin and/or F-actin in a variety of diagnostic assays. In various embodiments, antibodies or fragments thereof, whether labeled or unlabeled that bind specifically to G-actin and/or F-actin and/or TB4 are commercially available. Any immunoassay may use any antibodies, antibody fragments or derivatives thereof capable of binding the G-actin and/or F-actin molecules (e.g., monoclonal Abs, polyclonal Abs, Fab, F(ab′₂), Fv, or scFv fragments, or antigen binding fragments thereof). Such immunoassays are well-known in the art. In addition, if the G-actin and/or F-actin and/or TB4 expression is being measured as an mRNA polynucleotide, or portion thereof, it can be detected using nucleic acid hybridization techniques using well-established techniques.

### Methods for Screening Subjects

In various embodiments, the present invention provides methods for diagnosing and prognosing whether a subject who may have SIRS or may have recently experienced a trauma, burn, surgery or an infection, will progress to sepsis, remain in sepsis, likely to progress to septic shock or is in septic shock.

In one embodiment, the methods of the present invention for diagnosing or prognosing sepsis in a subject involve the measurement of G-actin and F-actin levels in the subject being diagnosed or prognosed. Actin is the most abundant protein in most eukaryotic cells and participates in numerous protein-protein interactions. Actin influences cell morphology, muscle contraction, and cell motility. It is present in two forms: a monomeric G-actin that can rapidly polymerize into its filamentous F-actin form. The intracellular pool of monomeric G-actin is divided into two groups: the large pool of sequestered monomeric G-actin, which is complexed to and regulated by actin binding proteins (ABPs) such as Thymosin Beta-4 (TB4) or Gelsolin, and a smaller pool of free monomeric actin that is in rapid equilibrium with filamentous actin. To the best of our knowledge, the concentration of G-actin and F-actin in the serum of healthy humans as well as patients in septic shock was unknown prior to the work conducted by the inventors.

TB4 is expressed in almost all eukaryotic cells. Its main intracellular activity is to bind G-actin into a 1:1 complex, rendering G-actin resistant to polymerization into its filamentous F-actin form. TB4 is important in maintaining a large intracellular volume of monomeric actin that is readily available for use if needed. TB4 has other activities such as preventing apoptosis by decreasing cytochrome c release from mitochondria, increasing bcl-2 expression, and decreasing caspase activation. It has currently passed phase 2 trials for severe dry eyes associated with graft versus host disease, pressure and venous stasis ulcers, and is being considered for phase 2 trials in peripheral neuropathy and stroke.

In murine sepsis models, TB4 levels have been shown to decrease when exposed to lipopolysaccharide from E. coli. Additionally, mice exposed to endotoxin-induced septic shock had decreased mortality when pre-treated with exogenous TB4, suggesting a role for TB4 and actin in the pathogenesis of sepsis.

In one embodiment, the methods of the present invention for diagnosing or prognosing sepsis in a subject comprising the steps: (a) providing a biological sample from the subject suspected of having sepsis or a subject likely to develop sepsis; (b) determining the expression level of G-actin and F-actin in the biological sample; and (c) correlating the ratio of G-actin expression and F-actin expression in the biological sample to known standards. In some embodiments, if the ratio of F-actin/G-actin are above a certain threshold, the subject is diagnosed or prognosed to have sepsis, or likely to develop septic shock, or is in septic shock.

In various embodiments, determining the ratio of F-actin to G-actin enables a clinician or medical professional, to stratify the severity of the sepsis and enable certain treatment options to be correlated to the severity of the sepsis or tailor treatment procedures to avert worsening the sepsis or rescuing the subject from the irreversible and life threatening pathology of septic shock. In this regard, the present invention provides a method for classifying a sepsis condition in a subject for determining the effective course of treatment, the method comprising: (a) providing a biological sample from the subject suspected of having sepsis or a subject likely to develop sepsis; (b) determining the expression level of F-actin and G-actin in the biological sample; and (c) correlating the ratio of F-actin expression and G-actin expression in the biological sample to known standards. In some embodiments, once the expression levels of F-actin and G-actin are determined from the biological sample, they may optionally be standardized to a known volume or commonly known measurement of value. The expression values (whether standardized or not) of F-actin and G-actin, in the biological sample can be computed into a ratio of F-actin over G-actin. The ratio of F-actin over G-actin in the biological sample, is then compared to the ratio of F-actin over G-actin obtained from a population of subjects who do not have sepsis, (e.g. healthy controls), or from patients with SIRS, or patients who have a preexisting condition or disorder at the time of correlating the ratio. The F-actin/G-actin ratio can then be used to determine the subject's clinical status with respect to sepsis, the likelihood of progressing to septic shock or diagnosis and prognosis of septic shock by correlating the ratio of F-actin expression and G-actin expression in the biological sample to known standard values of the ratio of F-actin over G-actin, obtained from healthy controls, or from patients with SIRS, or patients who have a preexisting condition or disorder at the time of correlating the ratio. In some embodiments, the method for diagnosing sepsis comprises determining whether the ratio of F-actin to G-actin exceeds a threshold of three standard deviations above the mean F-actin to G-actin ratio in healthy controls, and if the ratio of F-actin expression and G-actin expression in the biological sample is above said threshold, then the subject is diagnosed or prognosed as having sepsis.

In a further embodiment, a method for diagnosing or prognosing a likelihood that a patient will progress to septic shock comprises correlating the ratio of F-actin expression and G-actin expression in the biological sample to known standards. In this example, if the ratio of F-actin to G-actin in the biological sample exceeds a threshold of three standard deviations above a mean F-actin to G-actin ratio in subjects having a preexisting condition or disorder at the time of deriving the ratio, then the subject is diagnosed or prognosed as having sepsis likely to proceed to septic shock. A subject having a preexisting condition or disorder includes a subject having a preexisting condition or disorder at the time of correlating the F-actin/G-actin ratio, for example, a subject diagnosed with a bacterial or viral infection, a subject diagnosed with SIRS, or suspected of having SIRS, a subject having one or more symptoms of an inflammatory condition, a subject diagnosed with an autoimmune disease, a subject having a surgery performed less than 72 hours, a subject admitted for medical treatment as a result of a trauma, a subject admitted for medical treatment as a result of a burn, a premature neonatal subject, or a subject diagnosed with a cardiovascular disease. For a patient diagnosed or prognosed as having sepsis likely to proceed to septic shock, the treatment may include a hybrid approach comprising a conservative treatment plan along with an aggressive treatment plan depending on the difference or delta of the subject's ratio of F-actin to G-actin. If the values are significantly higher than the mean F-actin/G-actin ratio, then the treatment plan can be shifted to a more aggressive form, for example, a mixture of antibiotics, anti-inflammatory agents, plasmapheresis, high dose ultrafiltration, extracorporeal membrane oxygenation, selective cytopheresis, selective antigen removal, continuous renal replacement therapy, or combinations thereof.

In a related embodiment, a method for diagnosing or prognosing a subject in septic shock comprises correlating the ratio of F-actin expression and G-actin expression in the biological sample to known standards. In this example, if the ratio of F-actin to G-actin in the biological sample exceeds a threshold of six standard deviations above a mean F-actin to G-actin ratio in subjects having a preexisting condition or disorder at the time of deriving the ratio, then the subject is diagnosed or prognosed as in septic shock.

In one embodiment, the diagnostic and/or prognostic methods comprises the use of a ratio of F-actin and G-actin and these levels are compared to reference threshold levels obtained from healthy control expression levels or from SIRS patients or patients with a preexisting condition. Once the diagnosis is made, it can be confirmed using TB4 as a secondary biomarker, wherein if the level of TB4 is below a certain threshold, then the subject is confirmed as having sepsis, or likely to progress to septic shock or is in septic shock.

In another embodiment, the invention provides a method for classifying or stratifying a sepsis condition in a subject for the purpose of determining an effective course of treatment. In this related embodiment, the method includes the steps of: (a) providing a biological sample from the subject suspected of having sepsis or a subject likely to develop sepsis; (b) determining the expression level of F-actin and G-actin in the biological sample; and (c) correlating the ratio of F-actin expression and G-actin expression in the biological sample to a known standard. In the above exemplary method, if the ratio of F-actin to G-actin in the biological sample exceeds a threshold of three standard deviations above the mean F-actin to G-actin ratio in healthy controls, then the subject is treated with a conservative treatment comprising antibiotics, anti-inflammatories, and organ support. In another embodiment, if the ratio of F-actin to G-actin in the biological sample exceeds a threshold of six standard deviations above the mean F-actin to G-actin ratio in subjects having a preexisting condition or disorder at the time of deriving the ratio, then the subject is treated aggressively, for example, with plasmapheresis, high dose ultrafiltration, extracorporeal membrane oxygenation, selective cytopheresis, selective antigen removal, continuous renal replacement therapy, or combinations thereof.

In another embodiment, the methods of the present invention for diagnosing or prognosing sepsis in a subject comprising the steps: (a) providing a biological sample from the subject suspected of having sepsis or a subject likely to develop sepsis; (b) determining the expression level of F-actin and TB4 in the biological sample; and (c) correlating the levels of F-actin expression and TB4 expression in the biological sample to known standards. In some embodiments, if the level of F-actin is above a certain threshold, and the level of TB4 is below a certain threshold, the subject is diagnosed or prognosed to have sepsis, or likely to develop septic shock, or is in septic shock.

In various embodiments, determining level of F-actin and TB4 enables a clinician or medical professional, to stratify the severity of the sepsis and enable certain treatment options to be correlated to the severity of the sepsis or tailor treatment procedures to avert worsening the sepsis or rescuing the subject from the irreversible and life threatening pathology of septic shock. In this regard, the present invention provides a method for classifying a sepsis condition in a subject for determining the effective course of treatment, the method comprising: (a) providing a biological sample from the subject suspected of having sepsis or a subject likely to develop sepsis; (b) determining the expression level of F-actin and TB4 in the biological sample; and (c) correlating the level of F-actin expression and TB4 expression in the biological sample to known standards. In some embodiments, once the expression levels of F-actin and TB4 are determined from the biological sample, they may optionally be standardized to a known volume or commonly known measurement of value.

The expression values (whether standardized or not) of F-actin and TB4, in the biological sample can be compared to standard levels of F-actin and TB4 in healthy controls or from patients with SIRS, or patients who have a preexisting condition or disorder at the time of measurement of these biomarkers. The F-actin and TB4 levels can then be used to determine the subject's clinical status with respect to sepsis, the likelihood of progressing to septic shock or diagnosis and prognosis of septic shock. If the level of F-actin in the test subject's biological sample is above a certain threshold, as determined from healthy controls, or from patients with SIRS, or patients who have a preexisting condition or disorder and the levels of TB4 are below a predetermined threshold as determined from healthy controls, or from patients with SIRS, or patients who have a preexisting condition, then the subject is treated with a treatment regimen that accounts for the severity of the sepsis. In some embodiments, the method for diagnosing sepsis comprises determining whether the level of F-actin exceeds a threshold of three standard deviations above the mean F-actin level in healthy controls, and determining whether the level of TB4 is below a threshold of three standard deviations below the mean TB4 level in healthy controls, and if the level of F-actin expression in the biological sample is above said threshold, and the level of TB4 expression in the biological sample is below said threshold, then the subject is diagnosed or prognosed as having sepsis.

In some embodiments, the method for diagnosing a subject likely to progress to septic shock comprises determining whether the level of F-actin exceeds a threshold of three standard deviations above the mean F-actin level in a SIRS patient, or a patient with a preexisting condition, and determining whether the level of TB4 is below a threshold of three standard deviations below the mean TB4 level in a SIRS patient, or a patient with a preexisting condition, and if the level of F-actin expression in the biological sample is above said threshold, and the level of TB4 expression in the biological sample is below said threshold, then the subject is diagnosed or prognosed as likely to progress to septic shock.

In some embodiments, the method for diagnosing a subject in septic shock comprises determining whether the level of F-actin exceeds a threshold of six standard deviations above the mean F-actin level in a SIRS patient, or a patient with a preexisting condition, and determining whether the level of TB4 is below a threshold of six standard deviations below the mean TB4 level in a SIRS patient, or a patient with a preexisting condition, and if the level of F-actin expression in the biological sample is above said threshold, and the level of TB4 expression in the biological sample is below said threshold, then the subject is diagnosed or prognosed as being in septic shock.

In various embodiments, once the subject has been diagnosed or prognosed as either having sepsis, likely to progress to septic shock or is in septic shock, then the course of treatment can be determined for the subject based on the subject's diagnosis and/or prognosis. For example, if the level of F-actin expression in the biological sample of the test subject exceeds a threshold of three standard deviations above the mean F-actin level in healthy controls, and the level of TB4 is below a threshold of three standard deviations below the mean TB4 level in healthy controls, then the subject is diagnosed or prognosed as having sepsis. This test subject can be treated using a standard course of care, for example, anti-inflammatories, antibiotics and organ support.

Similarly, if the level of F-actin expression level in the subject's biological sample exceeds a threshold of three standard deviations above the mean F-actin expression level in a SIRS patient, or a patient with a preexisting condition, and the level of TB4 expression is below a threshold of three standard deviations below the mean TB4 expression level in a SIRS patient, or a patient with a preexisting condition, then the subject is diagnosed or prognosed as likely to progress to septic shock. This test subject can be treated using a hybrid course of care, for example, anti-inflammatories, antibiotics, organ support, plasmapheresis, high dose ultrafiltration, extracooreal membrane oxygenation, selective cytopheresis, selective antigen removal, continuous renal replacement therapy, or combinations thereof.

In other embodiments if the level of F-actin expression level in the subject's biological sample exceeds a threshold of six standard deviations above the mean F-actin expression level in a SIRS patient, or a patient with a preexisting condition, and the level of TB4 expression is below a threshold of six standard deviations below the mean TB4 expression level in a SIRS patient, or a patient with a preexisting condition, then the subject is diagnosed or prognosed as being in septic shock. This test subject can be treated using an aggressive course of care, for example, plasmapheresis, high dose ultrafiltration, extracorporeal membrane oxygenation, selective cytopheresis, selective antigen removal, continuous renal replacement therapy, or combinations thereof.

### Collection & Measurement of F- & G-Actin & TB4 Levels

According to one embodiment, the methods of the present invention comprise generating an F-actin/G-actin ratio from a biological sample taken or derived from a subject. In various embodiments, the biomarker profile is the relative amount of F-actin and G-actin and/or TB4 present in a biological sample taken from the subject under investigation. The biological sample may be, for example, a body fluid, for example, whole blood, plasma, serum, red blood cells, platelets, neutrophils, eosinophils, basophils, lymphocytes, monocytes, saliva, sputum, urine, cerebral spinal fluid, or a body tissue, for example, cells, a cellular extract, a tissue sample, a tissue biopsy, or any sample that may be obtained from a subject using techniques well known to those of skill in the art. In a specific embodiment, a ratio of F-actin/G-actin and/or TB4 is determined using one or more biological samples (preferably the same type of biological sample) collected from a subject at one or more separate time points. In another specific embodiment, a plurality of F-actin/G-actin ratios and/or expression levels of TB4 are generated using biological samples obtained from a subject at separate time points. In one example, these biological samples are obtained from the subject either once or, alternatively, on a daily basis, or more frequently, e.g., every 4, 6, 8 or 12 hours for a period of 1-5 days. In a specific embodiment, an F-actin/G-actin ratio or TB4 expression level is determined using biological samples collected from a single tissue type.

Similarly, the F-actin/G-actin ratio and/or TB4 expression level is determined in a reference or control biological sample. A “reference” or “control” can also be referred to as a “reference” sample. A reference sample can be generated from a biological sample taken at a particular time point in a subject that is a healthy control, i.e. a subject or from a pooled biological sample from subjects, that do not have sepsis, or SIRS, or an infectious disease, or an inflammatory disease, or are otherwise healthy. In some embodiments, a reference profile can be generated from a biological sample taken at a particular time point in a subject that has a preexisting condition, i.e. a subject or from a pooled biological sample from subjects, that are diagnosed with an infection, a subject having one or more symptoms of an inflammatory condition, a subject diagnosed with an autoimmune disease, a subject having a surgery performed less than 72 hours, a subject admitted for medical treatment as a result of a trauma, a subject admitted for medical treatment as a result of a burn, a premature neonatal subject, and a subject diagnosed with a cardiovascular disease. The reference profile, or plurality of reference profiles, can be used to establish threshold values for the levels of G-actin and/or F-actin and/or TB4 in a biological sample.

In addition, a reference profile can be in the form of a threshold value or series of threshold values. For example, a single threshold value can be determined by averaging the values of F-actin/G-actin ratio and/or TB4 from healthy controls, or from subjects with a preexisting condition. Similarly, a single or two or more threshold values can be determined by averaging the values of a series of F-actin/G-actin ratios and/or TB4 from healthy control(s) or subject(s) with a preexisting condition. Thus, a threshold value can have a single value or a plurality of values, each value representing a level of a specific F-actin/G-actin ratio, and/or TB4, detected in a biological sample, for example a fluid biological sample, such as blood, plasma, serum, or urine sample, e.g., of a healthy control or plurality of healthy controls, or a subject with SIRS, or a preexisting condition, or a plurality of subjects with SIRS or a preexisting condition.

### Methods of Detecting F-Actin, G-Actin and TB4 Protein in a Biological Sample

In specific embodiments of the invention, one or more ratios of F-actin and G-actin can be obtained by detecting proteins, for example, by detecting the expression product (e.g., a nucleic acid or protein) of F-actin and G-actin or post-translationally modified, or otherwise modified, or processed forms of such proteins. In one embodiment, the amount of F-actin in the biological sample is determined by detecting and/or analyzing F-actin or a portion thereof using any method known to those skilled in the art for detecting proteins including, but not limited to protein microarray analysis, immunohistochemistry and mass spectrometry. In various illustrative embodiments, the G-actin to be used in the various assays described herein, can be human G-actin monomer. In some illustrative examples, human G-actin can include G-actin monomer having an amino acid sequence or a portion thereof (i.e. the mature form of G-actin (375 aa) having an accession number from the NCBI database Accession No. NP_001092.1. NM_001101.3. (SEQ ID NO: 1) and having an mRNA polynucleotide sequence of NCBI database Accession No. NM_001101 (a 1852 bp mRNA) (SEQ ID NO: 2). F-actin is polymerized human G-actin with ATP.

In some illustrative embodiments, TB4 otherwise known as Thymosin beta-4, or (TMSB4X, FX, PTMB4, TB4X, or TMSB4) has a human protein sequence provided in NCBI database Accession No. NP_066932, version NP 066932.1, GI:11056061 (SEQ ID NO: 3), and a TB4 mRNA as provided in NCBI database Accession No. NM_021109 (SEQ ID NO: 4), the disclosures of which are incorporated herein by reference in its entirety. In one embodiment, a representative amino acid sequence of TB4 is: MSDKPDMAEIEKFDKSKLKKTETQEKNPLPSKETIEQEKQAGES (SEQ ID NO:5). In some embodiments, measurement of TB4 as used in the various methods and kits disclosed herein, can also include measurement of an acylated tetra peptide “Ac-SDKP” (positions 2-5 of SEQ ID NO: 5) that is a surrogate for the full length molecule of TB4. In addition, the measurement of TB4 as used in the various methods and kits disclosed herein, can also include measurement of LKKTET (positions 18-22 of SEQ ID NO: 5) that is a surrogate for the full length molecule of TB4.

Standard techniques may be utilized for determining the amount of the F-actin and G-actin, and TB4 proteins of interest present in a biological sample. For example, standard techniques can be employed using, e.g., immunoassays such as, for example Western blot, immunoprecipitation followed by sodium dodecyl sulfate polyacrylamide gel electrophoresis, (SDS-PAGE), immunocytochemistry, and the like to determine the amount of protein or proteins of interest present in a sample. One exemplary agent for detecting a protein of interest is an antibody capable of specifically binding to F-actin, and/or G-actin or a single antibody, capable of binding to both F-actin and G-actin, preferably an antibody detectably labeled, either directly or indirectly. An exemplary agent for detecting TB4 in a biological sample, is an antibody capable of specifically binding TB4, preferably an antibody detectably labeled, either directly or indirectly.

For such detection methods, if desired a protein from the sample to be analyzed can easily be isolated using techniques which are well known to those of skill in the art. Protein isolation methods can, for example, be such as those described in Harlow and Lane, 1988, Antibodies: A Laboratory Manual, Cold Spring Harbor Laboratory Press (Cold Spring Harbor, N.Y.), which is incorporated by reference herein in its entirety.

In certain embodiments, methods of detection of the protein or proteins of interest involve their detection via interaction with a protein-specific antibody. For example, antibodies directed to a protein of interest (e.g., a protein expressed from a gene described herein, e.g., a protein listed in). Antibodies can be generated utilizing standard techniques well known to those of skill in the art. In specific embodiments, antibodies can be polyclonal, or more preferably, monoclonal. An intact antibody, or an antibody fragment (e.g., scFv, Fab or F(ab′)₂) can, for example, be used. In some embodiments, exemplary antibodies useful in the methods of the present invention include: mouse monoclonal anti-actin antibody (Cat. No. MA1-744 (mAbGEa) Thermo Fisher Scientific Rockford, Ill. USA). In some embodiments, exemplary antibodies useful in the methods of the present invention include: mouse monoclonal anti-TB4 antibody (Cat. No. MABT77 EMD Millipore, Billerica, Mass. USA). Methods for quantifying F-actin and G-actin and TB4 are well known in the art. For example, the biological sample can be dialyzed against a stabilization buffer containing protease inhibitors ((0.1 M PIPES, pH 6.9, 30% glycerol, 5% DMSO, 1 mM MgSO₄, 1 mM EGTA, 1% TX-100, 1 mM ATP, and protease inhibitor) on ice for 2-6 hours). The retentate is then centrifuged in a tabletop centrifuge at 16.000 g. The supernatant containing G-actin is recovered, and the pellet containing F-actin was solubilized with actin depolymerization F-actin was solubilized with actin depolymerization buffer (0.1 M PIPES, pH 6.9, 1 mM MgSO₄, 10 mM CaCl₂, and 5 μM cytochalasin D). Aliquots of supernatant and pellet fractions are separated on 12% SDS-PAGE gels and then western blotted with monoclonal anti-actin antibody (mouse monoclonal anti-actin antibody (Cat. No. MA1-744 (mAbGEa) Thermo Fisher Scientific Rockford, Ill. USA). Signal for each actin sample is detected by ECL in a digital dark room and integrated optical band density can be used to estimate the cellular F/G-actin ratio. Commercial kits for determining the F/G actin ratio are also available, for example, the G-Actin/F-actin In Vivo Assay Biochem Kit (Cat. No. BK037, Cytoskeleton Inc. Denver, Colo. USA). Alternatively, G-actin and F-actin levels can be determined directly from the biological sample, optionally diluted with an appropriate buffer using an ELISA based immunoassay.

For example, antibodies, or fragments of antibodies, specific for F-actin and/or G-actin of interest can be used to quantitatively or qualitatively detect the presence of these proteins. This can be accomplished, for example, by an immune assay, such as an immunofluorescence, immunoprecipitation, western blotting, ELISA techniques, or combinations thereof. Antibodies (or fragments thereof) can, additionally, be employed histologically, as in immunofluorescence or immunoelectron microscopy, for in situ detection of F-actin and/or G-actin. In situ detection can be accomplished by removing a biological sample (e.g., a biopsy specimen) from a patient, and applying thereto a labeled antibody that is directed to F-actin and/or G-actin. The antibody (or fragment) is preferably applied by overlaying the antibody (or fragment) onto a biological sample. Through the use of such a procedure, it is possible to determine not only the presence of F-actin and/or G-actin, but also its distribution, in a particular sample. A wide variety of well-known histological methods (such as staining procedures) can be utilized to achieve such in situ detection.

Immunoassays for F-actin and/or G-actin typically comprise incubating a biological sample of a detectably labeled antibody capable of identifying F-actin and/or G-actin, and detecting the bound antibody by any of a number of techniques well-known in the art. As discussed in more detail, below, the term “labeled” can refer to direct labeling of the antibody via, e.g., coupling (i.e., physically linking) a detectable substance to the antibody, and can also refer to indirect labeling of the antibody by reactivity with another reagent that is directly labeled. Examples of indirect labeling include detection of a primary antibody using a fluorescently labeled secondary antibody.

The biological sample can be brought in contact with and immobilized onto a solid phase support or carrier such as nitrocellulose, or other solid support which is capable of immobilizing cells, cell particles or soluble proteins. The support can then be washed with suitable buffers followed by treatment with the detectably labeled antibody. The solid phase support can then be washed with the buffer a second time to remove unbound antibody. The amount of bound label on solid support can then be detected by conventional methods.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides and magnetite. The nature of the carrier can be either soluble to some extent or insoluble for the purposes of the present invention. The support material can have virtually any possible structural configuration so long as the coupled molecule is capable of binding to an antigen or antibody. Thus, the support configuration can be spherical, as in a bead, or cylindrical, as in the inside surface of a test tube, or the external surface of a rod. Alternatively, the surface can be flat such as a sheet, test strip, etc. Preferred supports include polystyrene beads. Those skilled in the art will know many other suitable carriers for binding antibody or antigen, or will be able to ascertain the same by use of routine experimentation.

One of the ways in which an antibody specific for F-actin and/or G-actin and/or TB4 can be detectably labeled is by linking the same to an enzyme and use in an enzyme immunoassay (EIA) (Voller, 1978, “The Enzyme Linked Immunosorbent Assay (ELISA)”, Diagnostic Horizons 2:1-7, Microbiological Associates Quarterly Publication, Walkeraville, Md.; Voller et al., 1978, J. Clin. Pathol. 31:507-520; Butler, J. E., 1981, Meth. Enzymol. 73:482-523; Maggio (ed.), 1980, Enzyme Immunoassay, CRC Press, Boca Raton, Fla.; Ishikawa et al., (eds.), 1981, Enzyme Immunoassay, Kgaku Shoin, Tokyo, each of which is hereby incorporated by reference in its entirety). The enzyme which is bound to the antibody will react with an appropriate substrate, preferably a chromogenic substrate, in such a manner as to produce a chemical moiety which can be detected, for example, by spectrophotometric, fluorimetric or by visual means.

Detection can also be accomplished using any of a variety of other immunoassays. For example, by radioactively labeling the antibodies or antibody fragments, it is possible to detect F-actin and/or G-actin and/or TB4 through the use of a radioimmunoassay (RIA).

It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethyletriaminpentcetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound can be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and acquorin. In another embodiment, specific binding molecules other than antibodies, such as aptamers, may be used to bind the biomarkers F-actin and/or G-actin and/or TB4.

In specific embodiments of the invention, relative G-actin levels and/or TB4 levels in the biological sample can be quantified using assays that detect and quantify nucleic acids encoding G-actin or TB4. Methods useful in the detection and quantification of RNA encoding G-actin or TB4 can be accomplished using any method well known to those skilled in the art including, hybridization, microarray analysis, RT-PCR, nuclease protection assays and Northern blot analysis.

In certain embodiments, nucleic acids encoding G-actin and/or TB4 can be detected and/or analyzed by the methods and compositions of the invention include RNA molecules such as, for example, expressed RNA molecules which include messenger RNA (mRNA) molecules, mRNA spliced variants as well as regulatory RNA, cRNA molecules (e.g., RNA molecules prepared from cDNA molecules that are transcribed in vitro) and discriminating fragments thereof.

The nucleic acid molecules detected and/or analyzed by the methods and compositions of the invention may be naturally occurring nucleic acid molecules such as RNA molecules, such as mRNA molecules, present in, isolated from or derived from a biological sample. The sample of nucleic acids detected and/or analyzed by the methods and compositions of the invention comprise, e.g., molecules of RNA, or copolymers of RNA. Generally, these nucleic acids correspond to particular genes or alleles of genes, or to particular gone transcripts (e.g., to particular mRNA sequences expressed in specific cell types. The nucleic acids detected and/or analyzed by the methods and compositions of the invention may correspond to different exons of the same gene, e.g., so that different splice variants of that gene may be detected and/or analyzed.

In specific embodiments, the nucleic acids are prepared in vitro from nucleic acids present in, or isolated or partially isolated from biological a sample. For example, in one embodiment, RNA is extracted from a sample (e.g., total cellular RNA, poly(A)⁺, messenger RNA, fraction thereof) and messenger RNA is purified from the total extracted RNA. Methods for preparing total and poly(A)⁺ RNA are well known in the art, and are described generally, e.g., in Sambrook et al., 2001, Molecular Cloning: A Laboratory Manual 3^{rd} ed. Cold Spring Harbor Laboratory Press (Cold Spring Harbor, N.Y.), which is incorporated by reference herein in its entirety. In one embodiment, RNA is extracted from a biological sample using guanidinium thiocyanate lysis followed by CsCl centrifugation and an oligo dT purification. In another embodiment, RNA is extracted from a sample using guanidinium thiocyanate lysis followed by purification on RNeasy columns (Qiagen, Valencia, Calif. USA). In one illustrative embodiment, the target nucleic acids are cRNA prepared from purified messenger RNA extracted from a sample. As used herein, cRNA is defined here as RNA complementary to the source RNA. The extracted RNAs are amplified using a process in which doubled-stranded cDNAs are synthesized from the RNAs using a primer linked to an RNA polymerase promoter in a direction capable of directing transcription of anti-sense RNA.

In one embodiment, to determine the level of G-actin and/or TB4 in the assays of the invention, the level of expression of G-actin and/or TB4 can be measured by amplifying RNA from a sample using reverse transcription (RT) in combination with the polymerase chain reaction (PCR). In accordance with this embodiment, the reverse transcription may be quantitative or semi-quantitative. Total RNA, or mRNA from a sample is used as a template and a primer specific to the transcribed portion of the gene(s) is used to initiate reverse transcription. Methods of reverse transcribing RNA into cDNA are well known and described in Sambrook et al., 2001, supra. Primer design can be accomplished based on known nucleotide sequences that have been published or available from any publicly available sequence database such as GenBank. The product of the reverse transcription is subsequently used as a template for PCR. PCR provides a method for rapidly amplifying a particular nucleic acid sequence by using multiple cycles of DNA replication catalyzed by a thermostable, DNA-dependent DNA polymerase to amplify the target sequence of interest. PCR requires the presence of a nucleic acid to be amplified, two single-stranded oligonucleotide primers flanking the sequence to be amplified, a DNA polymerase, deoxyribonucleoside triphosphates, a buffer and salts. The method of PCR is well known in the art. PCR is performed, for example, as described in Mullis and Faloona, 1987, Methods Enzymol. 155:335, which is hereby incorporated herein by reference in its entirety.

Quantitative RT-PCR (“QRT-PCR”), which is quantitative in nature, can also be performed to provide a quantitative measure of gene expression levels. In QRT-PCR reverse transcription and PCR can be performed in two steps, or reverse transcription combined with PCR can be performed concurrently. One of these techniques, for which there are commercially available kits such as Taqman (Perkin Elmer, Foster City, Calif. USA.) or as provided by Applied Biosystems (Foster City, Calif.) is performed with a transcript-specific antisense probe. This probe is specific for the PCR product (e.g. a nucleic acid fragment derived from a gene) and is prepared with a quencher and fluorescent reporter probe complexed to the 5′ end of the oligonucleotide. Different fluorescent markers are attached to different reporters, allowing for measurement of two products in one reaction. When Taq DNA polymerase is activated, it cleaves off the fluorescent reporters of the probe bound to the template by virtue of its 5′-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions are performed in 96-well plates so that samples derived from many individuals are processed and measured simultaneously. The Taqman system, has the additional advantage of not requiring gel electrophoresis and allows for quantification when used with a standard curve. A second technique useful for detecting PCR products quantitatively is to use an intercolating dye such as the commercially available QuantiTect SYBR Green PCR (Qiagen, Valencia, Calif. USA.). RT-PCR is performed using SYBR green as a fluorescent label which is incorporated into the PCR product during the PCR stage and produces a fluorescence proportional to the amount of PCR product.

Both Taqman and QuantiTect SYBR systems can be used subsequent to reverse transcription of RNA. Reverse transcription can either be performed in the same reaction mixture as the PCR step (one-step protocol) or reverse transcription can be performed first prior to amplification utilizing PCR (two-step protocol).

In some embodiments, other systems to quantitatively measure G-actin and/or TB4 mRNA expression products are known including Molecular Beacons which uses a probe having a fluorescent molecule and a quencher molecule, the probe capable of forming a hairpin structure such that when in the hairpin form, the fluorescence molecule is quenched, and when hybridized the fluorescence increases giving a quantitative measurement of gene expression. Other techniques to quantitatively measure RNA expression can include polymease chain reaction, ligase chain reaction, Qbeta replicase (see, e.g., International Application No. PCT/US87/00880, which is hereby incorporated by reference), isothermal amplification method (see, e.g., Walker et al., 1992, PNAS 89:382-396, which is hereby incorporated herein by reference), strand displacement amplification (SDA), repair chain reaction, Asymmetric Quantitative PCR and the multiplex microsphere bead assay.

In some embodiments, the level of expression of G-actin and/or TB4 can, for example, be measured by amplifying RNA from a sample using amplification (NASBA) wherein the nucleic acids may be prepared for amplification using conventional methods, e.g., phenol/chloroform extraction, heat denaturation, treatment with lysis buffer and minispin columns for isolation of DNA and RNA or guanidinium chloride extraction of RNA. These amplification techniques involve annealing a primer that has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once with a polymerase such as T7 or SP6. The resulting products, whether truncated or complete, indicate target G-actin specific sequences.

In one illustrative embodiment, quantification and measurement of G-actin and/or TB4 mRNA can be obtained by performing nuclease protection assays. Such assays are described in, for example, Sambrook et al., 2001, supra. In nuclease protection assays, an antisense probe (labeled with, e.g., radiolabeled or nonisotopic) hybridizes in solution to an RNA sample isolated from a subject's biological sample. Following hybridization, single-stranded, unhybridized probe and RNA are degraded by nucleases. An acrylamide gel is used to separate the remaining protected fragments. Typically, solution hybridization is more efficient than membrane-based hybridization, and it can accommodate up to 100 μg of sample RNA, compared with the 20-30 μg maximum of blot hybridizations. The ribonuclease protection assay, which is the most common type of nuclease protection assay, requires the use of RNA probes. Oligonucleotides and other single-stranded DNA probes can only be used in assays containing S1 nuclease. The single-stranded, antisense probe must typically be completely homologous to target RNA to prevent cleavage of the probe:target hybrid by nuclease.

In another illustrative embodiment, Northern Blot Assays can be used to identify and quantify G-actin and/or TB4 RNA. A standard Northern blot assay can be used to ascertain an RNA transcript size, identify alternatively spliced RNA transcripts, and the relative amounts of G-actin and/or TB4 RNA transcripts described herein (in particular, mRNA) in a sample, in accordance with conventional Northern hybridization techniques known to those persons of ordinary skill in the art. In Northern blots, RNA samples (e.g. from control, or SIRS subjects or subjects with a preexisting condition) are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. The probe can be labeled by any of the many different methods known to those skilled in this art. The labels most commonly employed for these studies are radioactive elements, enzymes, chemicals that fluoresce when exposed to ultraviolet light, and others. A number of fluorescent materials are known and can be utilized as labels. These include, but are not limited to, fluorescein, rhodamine, auramine, Texas Red, AMCA blue and Lucifer Yellow. The radioactive label can be detected by any of the currently available counting procedures. Enzyme labels are likewise useful, and can be detected by any of the presently utilized colorimetric, spectrophotometric, fluorospectophotometric, amperometric or gasometric techniques. The enzyme is conjugated to the selected particle by reaction with bridging molecules such as carbodiimides, diisocyanates, glutaraldehyde and the like. Any enzymes known to one of skill in the art can be utilized. Examples of such enzymes include, but are not limited to, peroxidase, beta-D-galactosidase, urease, glucose oxidase plus paeroxidase and alkaline phosphatase.

In some embodiments, RNA extraction, microarray hybridization, and microarray analysis can be used to determine the relative quantities of G-actin RNA in control and subject biological samples. In one illustrative embodiment, total RNA can be isolated from whole blood samples obtained from control and a patient's biological sample using the PaxGene Blood RNA System (PreAnalytiX, Qiagen/Becton Dickinson, Calif.) according the manufacturer's specifications. Microarray hybridization can be performed using any commercially available gene chip system, for example, the Affymetrix Gene Chip (Affimetrix Cleveland, Ohio USA).

In one illustrative embodiment, analyses of G-actin and/or TB4 in control or reference and patient samples can be performed using one patient or control sample per chip. Image files can be captured using an Affymetrix GeneChip Scanner 3000. CEL files produced can be subsequently preprocessed using Robust Multiple-array Average (RMA) normalization using GeneSpring GX 7.3 software (Agilent Technologies, Palo Alto, Calif.). All signal intensity-based data is used after RNA normalization, which specifically suppresses all but significant variation among lower intensity probe sets. All chips are then normalized to the respective median values of controls. Differences in G-actin and/or TB4 mRNA abundance between patient and control samples are determined using GeneSpring GX 7.3. All statistical analyses can be corrected for multiple comparisons. The specific statistical and filtering approaches can be modified in accordance to their relevance to data interpretation. F-actin levels derived from the same biological samples as used to determine the G-actin and/or TB4 expression levels using the microarray example above, can be determined using antibodies to F-actin using specific F-actin standard curves.

### Kits

The present invention further contemplates a kit or a diagnostic companion device or apparatus to enable the performance of the present methods described herein. In one embodiment, a kit can be used to assess the subject and determine whether the subject will remain in a pre-septic state, such as a SIRS subject, a subject with an infection or inflammatory condition that will not progress to sepsis or septic shock. In one illustrative embodiment, a kit of the present invention can be used to determine the likelihood of a patient with a preexisting condition to progress to sepsis and/or septic shock. Various embodiments of the present invention, kits utilize reagents that are able to detect and quantify the presence of G-actin and/or F-actin and/or TB4 in a subject's biological sample. In some embodiments, the kit may contain reagents that measure the expression and quantity of G-actin and F-actin and/or TB4 that enable the determination of an F-actin/G-actin ratio and/or levels of TB4 for use with the present methods described herein. As previously described, expression of F-actin and/or G-actin and/or TB4 can be performed using proteins, e.g. antibodies or fragments thereof, or nucleic acids, e.g. quantitative RT-PCT methods as illustrative examples.

In some embodiments, kits of the present invention may employ an antibody based system to determine F-actin/G-actin ratios and/or TB4. An antibody may be provided in a kit, which may include instructions for use of the antibody, e.g. in determining the presence of G-actin and/or F-actin and/or TB4 in a biological sample. One or more other reagents may be included, such as labeling molecules, buffer solutions, elutants and so on. Reagents may be provided within containers which protect them from the external environment, such as a sealed vial. The kit may include antibodies, fragments or derivatives thereof (e.g., Fab, F(ab′)₂, Fv, or scFv fragments) that are specific for F-actin and/or G-actin and/or TB4 of the present invention. In one embodiment, the antibodies may be detectably labeled.

In some embodiments, specific alterations in inflammatory and insulin resistance cytokines (IL-1-alpha, IL-Ip, IL-2, IL-4, TNF-alpha, TNF-R, IL-6, MCP-1, IL-8, IL-11, IL-12, and VCAM), and angiogenesis related growth factors (PlGF, FGF-2) and a growth factor antagonist (sFlt-1) can be used as secondary markers of sepsis and septic shock. In any event, these cytokines, chemokines, and angiogenesis related growth factors, or antagonists thereof, can be used to supplement the diagnostic or prognostic accuracy of the present invention.

The kits of the present invention may also include additional compositions, such as buffers, that can be used in constructing the F-actin/G-actin ratio. Prevention of the action of microorganisms can be ensured by the inclusion of various antibacterial and antifungal agents, for example, paraben, chlorobutanol, phenol sorbic acid, and the like. It may also be desirable to include isotonic agents such as sugars, sodium chloride, and the like.

Some kits of the invention may further comprise a computer program product for use in conjunction with a computer system, wherein the computer program product comprises a computer readable storage medium and a computer program mechanism embedded therein. In such kits, the computer program mechanism comprises instructions for evaluating whether a one or more ratios of the levels of expression of F-actin and G-actin of a test subject at risk for developing sepsis satisfies a threshold level of a healthy reference sample or a subject with a preexisting condition sample. Satisfying the first threshold value with respect to healthy controls predicts that the test subject is likely to develop sepsis. In some embodiments, if the ratio of F-actin to G-actin exceeds a threshold of three standard deviations above the mean F-actin to G-actin ratio in healthy controls, and if the ratio of F-actin expression and G-actin expression in the biological sample of the test subject is above the threshold, then the subject is diagnosed or prognosed as having sepsis. In another embodiment, the computer program mechanism comprises instructions for evaluating whether one or more ratios of the levels of expression of F-actin and G-actin of a test subject is at risk for progressing to septic shock, or is in septic shock satisfies. The computer program mechanism may use a threshold level of a reference sample from a subject or plurality of subjects with a preexisting condition sample that is different from the reference value obtained from healthy controls. Satisfying the conditions above the threshold value with respect to reference sample ratios of F-actin/G-actin from subjects with a preexisting condition, predicts that the test subject is likely to progress to septic shock or is in septic shock. In some embodiments, if the ratio of F-actin to G-actin in a test subject biological sample exceeds a threshold of three or six standard deviations above the mean F-actin to G-actin ratio in subjects with a preexisting condition, the test subject is diagnosed or prognosed as likely to progress to septic shock or is in septic shock respectively.

Some kits of the present invention comprise a computer having a central processing unit and a memory coupled to the central processing unit. The memory stores instructions for evaluating whether a ratio of F-actin over G-actin (F-actin/G-actin) of a test subject at risk for developing sepsis, likely to progress to septic shock or is in septic shock.

### Methods of Treatment of Subjects Suspected of Having Severe Sepsis or Septic Shock

The present invention also provides for methods for treating a subject that presents to a medical provider, such as a hospital or a medical provider assessing whether a subject who has a preexisting condition should be treated with a standard protocol of care for a SIRS patient, or a patient suspected of having sepsis or should be treated with an aggressive form of therapy to prevent or ameliorate one or more symptoms of septic shock.

In various embodiments, the subject under evaluation, i.e. the test subject, will have a biological sample obtained and assessed to determine the ratio of F-actin over G-actin (F-actin/G-actin) and optionally TB4 using the methods described above. Once the determination of the F-actin/G-actin ratio and/or TB4 is obtained, the F-actin/G-actin ratio is compared to a control or reference sample F-actin/G-actin ratio. The reference or control sample can be a healthy control reference sample or a preexisting condition reference or control sample. The test subject's F-actin and G-actin ratio is then determined whether it exceeds (i.e. is higher than) the threshold of three standard deviations above the mean F-actin to G-actin ratio in healthy control or reference subjects. If the ratio of F-actin expression and G-actin expression in the biological sample of the test subject is above the threshold, then the test subject is diagnosed or prognosed as having sepsis. This test subject can be treated using a standard course of care, for example, anti-inflammatories, antibiotics and organ support. In some embodiments, a subject that presents at a medical facility, for example, a subject with an infection, an inflammatory condition, or SIRS is diagnosed whether the subject has sepsis or is likely to progress to septic shock by first obtaining a biological sample. The biological sample is then used to assess whether the subject has an F-actin/G-actin ratio that is above a threshold of F-actin/G-actin ratio in healthy controls, and/or patients with a preexisting condition, for example, SIRS. In one embodiment, if the subject presents to the medical facility and is clinically diagnosed with SIRS, the diagnostic and prognostic methods of the present invention can be used to determine whether the SIRS subject is likely to have sepsis, or likely to progress to septic shock. In the event that the subject is likely to have sepsis but not confirmed with a positive culture, the subject can be started with antibiotic therapy, in advance of a positive culture.

Similarly, the test subject's F-actin and G-actin ratio is then determined whether it exceeds (i.e. is higher than) the threshold of three standard deviations above the mean F-actin to G-actin ratio of subjects with a preexisting condition. If the ratio of F-actin expression and G-actin expression in the biological sample of the test subject is above the threshold at the time of determining the ratio, then the test subject is diagnosed or prognosed as likely to proceed to septic shock. This test subject can be treated using a hybrid course of care, for example, anti-inflammatories, antibiotics, organ support, plasmapheresis, high dose ultrafiltration, extracorporeal membrane oxygenation, or combinations thereof.

In other embodiments, the test subject's F-actin and G-actin ratio is determined whether it exceeds (i.e. is higher than) the threshold of six standard deviations above the mean F-actin to G-actin ratio of subjects with a preexisting condition. If the ratio of F-actin expression and G-actin expression in the biological sample of the test subject is above the threshold at the time of determining the ratio, then the test subject is diagnosed or prognosed as having septic shock. This test subject can be treated using an aggressive course of care, for example, plasmapheresis, high dose ultrafiltration, extracorporeal membrane oxygenation, selective cytopheresis, selective antigen removal, continuous renal replacement therapy, or combinations thereof.

In the present disclosure, the terms “sepsis”, “severe sepsis” and “septic shock” are to be understood according to the definitions established in 1991 by the American College of Chest Physicians (ACCP) and the Society of Critical Care Medicine (SCCM) and confirmed in 2001 (Levy et al., 2003). These and other definitions used herein are summarized below: A “sepsis” is a systemic inflammation in response to infection. A “severe sepsis” is defined as a sepsis with at least one organ dysfunction. Among severe sepsis syndromes, the most severe cases exhibit two organ failures or even more. Although not very common, some severe sepsis cases with two organ dysfunctions distinct from acute respiratory failure are observed: this kind of cases are defined as “severe sepsis with at least two organ failures”, and are distinct from septic shocks. A “septic shock” is defined as a sepsis with acute circulatory failure. An “acute circulatory failure” is a persistent arterial hypotension (systolic arterial pressure <90 mm H& a MAP<60 mmHg or a reduction in systolic blood pressure of >40 mm Hg from baseline) despite adequate volume resuscitation, in the absence of other causes for hypotension. A “septic shock with at least two organ failures” is a septic shock with at least one organ failure (e.g., kidney, liver, or brain) in addition to the acute circulatory failure. In the present disclosure, patients who are considered are those, either non-infectious SIRS or infectious SIRS. For these patients, “day 0” designates the 24-hours period after the onset of at least one SIRS criteria.

A first aspect of the present invention is a method for treating or preventing septic shock in a non-infectious SIRS subject or an infectious SIRS subject, the method comprising: (a) obtaining a blood sample from the non-infectious SIRS subject or an infectious SIRS subject; (b) determining the amount of F-actin in the non-infectious SIRS subject or an infectious SIRS subject blood sample; (c) determining that the non-infectious SIRS subject or the infectious SIRS subject is in septic shock if the non-infectious SIRS subject or the infectious SIRS subject's F-actin level is about 3 ng/mL or greater, and (d) administering an effective treatment to treat or prevent septic shock in the non-infectious SIRS subject or the infectious SIRS subject having an F-actin level of about 3 ng/mL or greater.

According to some embodiments of this method, the blood sample used in step (a) has been collected at day 0, day 1 or day 2 after the onset of SIRS, or when admitted for treatment. In another embodiment, the blood sample has been collected at day 0 or the same day the symptoms of SIRS has occurred. The blood sample can be, for example, selected amongst whole blood, plasma or serum, or combinations thereof preferably plasma.

The threshold to be considered when performing the above method is predetermined by measuring the level of F-actin in a representative cohort of individuals having undergone a severe sepsis or septic shock, and for whom the outcome is known. The threshold is calculated to obtain the best predictability (sensitivity and specificity) for the risk of developing septic shock or not. For example, when the level of F-actin is measured in the blood or plasma with a technology similar to that described herein, a predetermined threshold of about 3 ng/mL, for example, about 2.9 ng/mL, or about 3.0 ng/mL, or about 3.1 ng/mL, or about 3.2 ng/mL, or about 3.3 ng/mL, or about 3.4 ng/mL, or about 3.5 ng/mL or greater level of F-actin can be considered. On the cohort used in the example section, the level of F-actin of about 3 ng/mL or higher led to a specificity of prognosis (risk of developing septic shock) of 100%, considering this threshold. Of course, the skilled artisan is free to re-evaluate this threshold on a larger cohort of patients, and by using any kind of technology for measuring the F-actin level in a subject with either a non-infectious SIRS or an infectious SIRS status at days 0, 1, or 2 or when admitted to medical care.

In a preferred embodiment, the measurement performed in step (i) is done by an immunoassay, for example with an antibody which specifically binds to F-actin, for example, human F-actin. Several examples of antibodies specifically binding to F-actin have been described herein. The skilled artisan can also use, instead of antibodies specific for F-actin, any other molecule specifically binding to F-actin, such as, for example, antibody fragments or specifically designed aptamers. Aptamers are single stranded nucleic acid molecules (DNA or RNA) that are selected in vitro for their ability to bind to a target molecule; this selection can be performed, for example, by the SELEX method (Systematic Evolution of Ligands by Exponential Enrichment) described in U.S. Pat. No. 5,270,163. In various embodiments, F-actin, for example, human F-actin, can be used by the skilled artisan for obtaining molecules specifically binding to the protein molecule. In various embodiments, an immunoassay can be prepared using a standard curve of F-actin for use in determining unknown concentrations of F-actin in a test sample.

In a particular embodiment of the method according to the present invention, the immunoassay performed is an ELISA assay such as ELISA assays described in the experimental part below. Alternatively, fluorescently labeled antibodies can be used, for example for performing flow cytometry, or any other immunoassays capable of determining an unknown concentration of F-actin. Of course, the skilled artisan can choose any other immunoassay for performing a method according to the present invention.

In some embodiments, wherein administering an effective treatment to treat or prevent septic shock in the non-infectious SIRS subject or the infectious SIRS subject, the method comprises treating the non-infectious SIRS subject or the infectious SIRS subject with plasmapheresis, high dose ultrafiltration, extracorporeal membrane oxygenation, selective cytopheresis, selective antigen removal, continuous renal replacement therapy, or combinations thereof. In certain embodiments, an infectious or non-infectious SIRS patient diagnosed with a blood or plasma F-actin value greater than about 3 ng/mL is diagnosed with a severe syndrome, for example, severe sepsis or septic shock and can be treated with one or more of the following interventions:
a. Administration of broad spectrum antibiotics;
b. Administration of IV Vancomycin loading dosage of 15-25 mg/kg and IV Zosyn 4.5 grams IV Q8 hours;
c. Administration of IV Vancomycin loading dosage of 15-25 mg/kg IV Cefepime 1-2 grams Q8 hours, and IV Flagyl 500 mg Q8 hours;
d. Administration of Intravenous fluid to maintain mean arterial pressure of 65 mm Hg or above, or fluid resuscitation based on other parameters (example: inferior vena cava collapsibility);
e. Administration of 2 liters of 0.9% NaCl;
f. Administration of Albumin 25-50 grams;
g. Administration of Actin Binding Protein;
h. Administration of Thymosin Beta 4; L Administration of Gelsolin;
j. Administration of vasopressor therapy to maintain a mean arterial pressure of 65 mm Hg or above;
k. Administration of IV Norepinephrine 0.01-3.0 mcg/kg/min; L Administration of IV Epinephrine 0.1-0.8 μg/kg/min;
m. Administration of Vasopressin 0.03 units/minute;
n. Administration of Dopamine 2-50 μg/kg/min;
o. Administration of ionotropic therapy;
p. Administration of dobutamine up to 20 micrograms/kg/minute if evidence of myocardial dysfunction or signs of hypoperfusion despite adequate intravascular volume and adequate mean arterial pressure;
q. Administration of packed red blood cell transfusion: Consideration of 1-2 U or packed red blood cell transfusion in a patient with a hemoglobin below 7.0 g/dL;
r. Administration of an arterial line for blood pressure monitoring;
s. Administration of a central line;
t. Maintenance of central venous oxygen to an oxygen saturation 70% or above.
u. Maintenance of central venous pressure 8-12 mm Hg.
v. Administration of corticosteroids
w. Administration of IV hydrocortisone 50-100 mg;

In various embodiments, treating or preventing severe sepsis or septic shock in a non-infectious SIRS subject or an infectious SIRS subject with a blood or plasma level of F-actin above about 3 ng/mL with any of the above interventions can also include the addition of therapeutically effective amounts of Thymosin-beta-4 (TB4) and/or a therapeutically effective amount of gelsolin (human plasma isoform).

In some embodiments, when a non-infectious SIRS or infectious SIRS patient is found to have a blood or plasma level of about 3 ng/mL or greater, the subject is treated by administering an effective treatment to treat or prevent septic shock in the non-infectious SIRS subject or the infectious SIRS subject. In various embodiments, the method comprises treating the non-infectious SIRS subject or the infectious SIRS subject with a blood or plasma level of F-actin above about 3 ng/mL with therapeutically effective amounts of Thymosin-beta-4 (TB4) and/or a therapeutically effective amount of gelsolin (human plasma isoform). Each of these active agents are commercially available, for example, human recombinant gelsolin is commercially available from Cytoskeleton Inc., (Denver, Colo., USA). Gelsolin can be used at therapeutically effective amounts ranging from about 0.1 mg/kg patient weight to about 100 mg/kg patient weight and all ranges therebetween, preferably from about 1 mg/kg, or about 3 mg/kg to about 6 mg/kg or about 10 mg/kg and all ranges therebetween. Human recombinant TB4 is commercially available from Advanced ChemTech, Inc. (Louisville, Ky., USA), at a specific activity of 5 mg/1000 U. Therapeutically effective amounts of TB4 can range from at least about 1 ng/mL, usually at least about 10 ng/mL, more usually at least about 100 ng/mL, and not more than about 10 μg/mL, more usually not more than about 1 μg, and may be used at a concentration of about 0.1 to 0.5 μg/mL.

In some embodiments, the skilled artisan can combine several markers for establishing a prognosis in cases of sepsis or septic shock. Amongst the markers which can be used in combination with F-actin concentration, G-actin and TB4 as described above can be used. The present invention hence also pertains to a method as described above, further comprising a step of measuring the level of at least one of G-actin and TB4 in a blood sample from the patient (the same biological sample as that in which F-actin concentration is measured, or another biological sample if appropriate), and a step of comparing said level to a predetermined threshold.

According to another aspect of the present invention, the invention also provides a method for performing a follow-up of a patient that has progressed passed SIRS and is now exhibiting symptoms of sepsis by measuring the evolution of the plasma level of F-actin in the patient, wherein a decrease in the level of F-actin indicates that the patient is recovering. According to this method, if a patient had a level of F-actin at DO above the predetermined threshold defined above, and if the level remains above the threshold after day 3, 4, 5, 6, or 7 this indicates that the patient has a great probability of death.

Another method according to the present invention aims at performing a follow-up of a patient in sepsis or in septic shock, by measuring the evolution of the expression level of F-actin in the patient, wherein a decrease in the level of F-actin indicates that the patient is recovering. When performing this method, the circulating levels of F-actin can be measured by any F-actin detection assay disclosed herein, for example, by immunoassays, for example, ELISA.

In the above-described follow-up methods, the measures of F-actin are performed on blood samples obtained from the patient at several time points after admission, for example each day during the first week and then, depending on the clinical context, at the same frequency or at a lower frequency.

According to yet another aspect, the present invention pertains to a method for helping decision for treatment withdrawal for a patient in severe sepsis with at least two organ failures or in septic shock with at least two organ failures, comprising the following steps: (i) establishing a prognosis for the patient, by a method according to the prognosis of developing septic shock described above; (ii) measuring the level of F-actin in the subject's blood sample, obtained after several days (e.g., 7 to 14 days) of treatment; wherein if no decrease in the level of F-actin in the subject's blood is observed and if the clinical status remains severe, treatment withdrawal is decided. When performing this method, the physician will consider that the clinical status remains severe if the patient still has two organ failures or more. Treatment withdrawal will in particular be decided if the F-actin blood level measured in step (i) was above the above-defined threshold and remains above this threshold after several days of treatment.

Since the present invention provides a reliable prognosis marker for patients in very severe conditions (i.e., severe sepsis or septic shock), this prognosis marker can be used to better select the individuals to be enrolled in clinical trials for testing new treatments aiming at improving either the duration of intensive support before the patient leaves the intensive care unit or the outcome of these pathologies.

In the first case, the patients who will be enrolled are those with a good prognosis, in order to avoid noise related to “desperate” patients. The invention hence also pertains to a method for determining if a subject in a very severe condition (i.e. with severe sepsis or septic shock) is to be enrolled in a clinical trial for evaluating the efficiency of a pharmaceutical treatment for shortening the need of intensive support for such patient, wherein said method comprises a step of establishing a prognosis for the subject by a method as described above, and wherein the subject is enrolled if the measured level of F-actin is below the predetermined threshold.

In the alternative, patients with a bad prognosis can be enrolled in trials for evaluating new treatments for improving outcome of very severe conditions i.e. severe sepsis and septic shock, so that a drug with potential severe side-effects will not be given to patients supposed to recover by “classical” resuscitation, and so that the results be free of noise related to patients who would have recovered without this new drug or treatment. Hence, the present invention also relates to a method for determining if a subject in severe sepsis or in septic shock is to be enrolled in a clinical trial for evaluating the efficiency of a pharmaceutical treatment for improving outcome for such a patient, comprising a step of establishing a prognosis for the subject by a method as above-described, wherein said subject is enrolled if the measured level of F-actin is above the predetermined threshold.

As a corollary of the above method, the invention also pertains to a method for testing the efficiency of a pharmaceutical treatment for improving outcome of severe syndromes, comprising the following steps: (i) selecting a patient in severe sepsis or in septic shock, and determining the level of F-actin in a blood sample from the patient obtained before the beginning of said pharmaceutical treatment; (ii) from at least another blood sample from the patient, obtained after the beginning of the pharmaceutical treatment, determining the level of F-actin in the patient's blood sample; (iii) comparing the obtained values; wherein a decrease in the blood F-actin level following the beginning of the pharmaceutical treatment indicates that said treatment has been beneficial to the patient and is likely to improve outcome of the severe syndromes. In various embodiments, the severe syndromes are severe sepsis and septic shock.

In an illustrative embodiment of the above-method, step (i) is performed at day 0 after the onset of severe sepsis or septic shock, and the selected patient preferably has a blood or plasma level of F-actin above a predetermined threshold of about 3 ng/mL. In this latter case, the treatment will be considered as beneficial to the patient and most likely to improve outcome of severe syndromes if the blood or plasma F-actin level decreases below the threshold of about 3 ng/mL.

Other characteristics of the invention will also become apparent in the course of the description which follows of the biological assays which have been performed in the framework of the invention and which provide it with the required experimental support, without limiting its scope.

### EMBODIMENTS

1. A method for diagnosing or prognosing sepsis in a subject comprising the steps:

(a) providing a biological sample from the subject suspected of having sepsis or a subject likely to develop sepsis;

(b) determining the expression level of G-actin and F-actin in the biological sample; and

(c) correlating the ratio of F-actin expression and G-actin expression (F-actin/G-actin) in the biological sample to a known standard.

2. The method of embodiment 1, wherein the biological sample comprises a body fluid, a tissue sample, a cell culture fluid, a cell lysate, or combinations thereof.

3. The method of embodiment 2, wherein the body fluid is selected from the group consisting of: blood, serum, plasma, urine, lymph, saliva, amniotic fluid, prostatic fluid, seminal fluid, biopsy fluid, gastrointestinal fluid, vaginal fluid and combinations thereof.

4. The method of embodiment 1, wherein the prognosing of sepsis comprises prognosis of sepsis, prognosis of sepsis likely to progress to septic shock, and prognosis of septic shock.

5. The method of embodiment 1, wherein the expression level of G-actin comprises, the amount of G-actin protein in the biological sample, the amount of G-actin mRNA in the biological sample, and combinations thereof.

6. The method of embodiment 5, wherein the G-actin protein comprises at least 85% of the amino acid sequence of SEQ ID NO: 1

7. The method of embodiment 5, wherein the G-actin mRNA comprises at least 85% of the nucleotide sequence SEQ ID NO: 2.

8. The method of embodiment 1, wherein the expression level of F-actin comprises, the amount of F-actin protein in the biological sample, the amount of F-actin mRNA in the biological sample, and combinations thereof.

9. The method of embodiment 5, wherein the F-actin protein comprises a polymer of G-actin having at least 85% of the amino acid sequence of SEQ ID NO: 3

10. The method of embodiment 5, wherein the G-actin protein comprises at least 95% of the amino acid sequence of SEQ ID NO: 4.

11. The method according to embodiment 1, wherein determining the expression level of F-actin and G-actin in the biological sample comprises using an agent independently selected from the group consisting of an antibody that binds to F-actin, an antibody that binds to G-actin, a F-actin binding partner, a G-actin binding partner, a nucleic acid that hybridizes to a nucleic acid encoding F-actin, and a nucleic acid that hybridizes to a nucleic acid encoding G-actin.

12. The method according to embodiment 1, wherein determining the expression level of F-actin and G-actin in the biological sample comprises determining the amount of F-actin and G-actin using an immunoassay.

13. The method according to embodiment 1, wherein determining the expression level of F-actin and G-actin in the biological sample comprises determining the amount of F-actin and G-actin using a nucleic acid hybridization assay.

14. The method according to embodiment 1, wherein correlating the ratio of F-actin expression and G-actin expression in the biological sample to known standards comprises determining whether the ratio of F-actin to G-actin exceeds a threshold of three standard deviations above the mean F-actin to G-actin ratio in healthy controls, and if the ratio of F-actin expression and G-actin expression in the biological sample is above said threshold, then the subject is diagnosed or prognosed as having sepsis.

15. The method according to embodiment 1, wherein correlating the ratio of F-actin expression and G-actin expression in the biological sample to known standards comprises determining whether the ratio of F-actin to G-actin exceeds a threshold of three standard deviations above the mean F-actin to G-actin ratio in subjects having a preexisting condition or disorder at the time of said correlating said ratio, and if the ratio of F-actin expression and G-actin expression in the biological sample is above said threshold, then the subject is diagnosed or prognosed as having sepsis likely to proceed to septic shock.

16. The method according to embodiment 15, wherein a subject having a preexisting condition or disorder at the time of correlating said ratio comprises, a subject diagnosed with an infection, SIRS, a subject having one or more symptoms of an inflammatory condition, a subject diagnosed with an autoimmune disease, a subject having a surgery performed less than 72 hours, a subject admitted for medical treatment as a result of a trauma, a subject admitted for medical treatment as a result of a burn, a premature neonatal subject, and a subject diagnosed with a cardiovascular disease.

17. The method according to embodiment 1, wherein correlating the ratio of F-actin expression and G-actin expression in the biological sample to known standards comprises determining whether the ratio of F-actin to G-actin exceeds a threshold of six standard deviations above the mean F-actin to G-actin ratio in subjects having a preexisting condition or disorder at the time of said correlating said ratio, and if the ratio of F-actin expression and G-actin expression in the biological sample is above said threshold, then the subject is diagnosed or prognosed as having septic shock.

18. A method for classifying a sepsis condition in a subject for determining an effective course of treatment, the method comprising:

(a) providing a biological sample from the subject suspected of having sepsis or a subject likely to develop sepsis;

(b) determining the expression level of F-actin and G-actin in the biological sample; and

(c) correlating the ratio of F-actin expression and G-actin expression in the biological sample to a known standard.

19. The method according to embodiment 18, wherein if the ratio of F-actin to G-actin in the biological sample exceeds a threshold of three standard deviations above the mean F-actin to G-actin ratio in healthy controls, then the subject is treated with antibiotics, anti-inflammatory, organ support, or combinations thereof.

20. The method according to embodiment 18, wherein if the ratio of F-actin to G-actin in the biological sample exceeds a threshold of six standard deviations above the mean F-actin to G-actin ratio in subjects having a preexisting condition or disorder at the time of said correlating said ratio, then the subject is treated with plasmapheresis, high dose ultrafiltration, extracorporeal membrane oxygenation, selective cytopheresis, selective antigen removal, continuous renal replacement therapy, or combinations thereof.

### EXAMPLES

### Example 1. Assays for Determining F-Actin and G-Actin in Healthy and Septic Shock Subjects

Introduction

Thymosin Beta 4 (TB4), a G-actin sequestering protein, inhibits the polymerization of monomeric G-actin into its polymeric form F-actin, thus influencing the formation of actin cytoskeleton and many other cellular functions. Exogenous TB4 has been shown to reduce lethality and down-regulate inflammatory mediators in a murine endotoxin-induced sepsis model. The inventors investigated the levels of TB4, G-actin and F-actin in the plasma of humans with septic shock over the first seven days of their hospitalization and compare them to healthy controls.

Introduction

Sepsis is the pathologic systemic inflammatory response to an infection. It is defined clinically as a suspected or known infection in the presence of two or more systemic inflammatory response syndrome (SIRS) criteria. SIRS criteria include elevated heart rate, elevated respiratory rate, elevated/decreased temperature, and/or elevated/decreased white blood count. In some cases, when a patient remains hypotensive despite two liters of intravenous fluids, the syndrome is defined as septic shock. The pathogenesis is thought to be multifactorial and a single trigger causing this deadly cascade is not known.

Despite advances in medical technology, the incidence of sepsis in developing countries is rising and accounts for major morbidity and mortality, associated with 36.9% to 55.9% of all inpatient mortalities. It is a major economic burden in America resulting in 20.3 billion dollars in total health care costs in 2013.

Actin is the most abundant protein in most eukaryotic cells and participates in numerous protein-protein interactions. Actin influences, cell morphology, muscle contraction, and cell motility. It is present in two forms: a monomeric G-actin that can rapidly polymerize into its filamentous F-actin form. The intracellular pool of monomeric G-actin is divided into two groups: the large pool of sequestered monomeric G-actin, which is complexed to and regulated by actin binding proteins (ABPs) such as Thymosin Beta-4 (TB4) or Gelsolin, and a smaller pool of free monomeric actin that is in rapid equilibrium with filamentous actin. To the best of our knowledge, the concentration of G-actin and F-actin in the serum of healthy humans as well as patients in septic shock was unknown prior to this study.

TB4 is expressed in almost all eukaryotic cells. Its main intracellular activity is to bind G-actin into a 1:1 complex, rendering G-actin resistant to polymerization into its filamentous F-actin form. TB4 is important in maintaining a large intracellular volume of monomeric actin that is readily available for use if needed. TB4 has other activities such as preventing apoptosis by decreasing cytochrome c release from mitochondria, increasing bcl-2 expression, and decreasing caspase activation. It has currently passed phase 2 trials for severe dry eyes associated with graft versus host disease, pressure and venous stasis ulcers, and is being considered for phase 2 trials in peripheral neuropathy and stroke.

To the best of the inventors' knowledge, levels of actin have never been quantified in sepsis or healthy patients. The aim of this study was to characterize serum levels of G-actin, F-actin, and TB4 in patients with septic shock and compare them with healthy controls, with the goal of elucidating possible etiologic mechanisms of septic shock that may lead to novel therapies.

Methods:

This study was performed in accordance with the ethical guidelines of Henry Ford Hospital and the study was approved by the Institutional Review Board. Written consent was obtained from healthy controls but was waived for patients in the septic shock group.

Serum samples were drawn from 26 patients diagnosed with septic shock and analyzed over three time points during their hospitalization (day zero, day three, and day seven). Levels of TB4, G-actin, and F-actin were measured in each serum sample. Seventeen healthy volunteers served as controls and samples were measured at one time point. Levels of each molecule were measured using enzyme-linked immunosorbent assays. A univariate Cox proportional hazard model was employed to determine the effect of time on each molecule studied. A Wilcoxon two-group test was used to study the medians of the septic versus controls groups.

A single whole blood sample was obtained from each of the 17 healthy controls and collected into an EDTA-containing tube. Within ten minutes of collection, samples were centrifuged at 2000 RPM for ten minutes and plasma was harvested. Specimens were then stored at −80° C. until analysis.

Serum samples were stored at −80° C. and used in enzyme-linked immunosorbent assays (ELISA) to measure TB4 (Wuxi Donglin Sci&Tech Development Co, Jiangsu, China), F-actin (MyBiosource, San Diego, Calif.), and G-actin (MyBiosource, San Diego, Calif.). Samples were assayed separately for each analyte following the manufacturer's instructions. Sample concentrations were derived from plotting ODs of standards to create a standard curve (TB4) or from a four parameter logistic curve (4-PL) (F-actin and G-actin), using Graph Pad Prism 4 v 5.04. Values below the lowest standard were reported as half the value of the lowest standard control while samples with values greater than the highest standard were reported as value for the highest standard control. The reporting ranges for the different analytes were TB4 (39 ng/mL-10,000 ng/mL), F-actin (0.31 ng/mL-40 ng/mL), G-actin (0.25 ng/mL-40 ng/mL). Due to previous data not published, the G-actin assay for the sepsis group was diluted 1:4 secondary to elevated levels found in this group.

All statistical analysis was performed with SAS 9.4, (SAS Institute Inc., Cary, N.C., USA) by an independent statistician. Because some values were below the limit of detection (LOD), the standard practice of using half the LOD was used (Example: LOD for F-actin-0.625 replaced by 0.3125). When values were greater than the LOD, their actual value could theoretically be infinity, thus they remained at their upper bound (40 μg/mL for G-actin sepsis group and 10 μg/mL for G-actin control). When values were outside of their LOD, they were considered censored.

A Wilcoxon two-group test was used to study the medians of the septic shock versus control groups. Results are reported in median (IQR) with a P value under 0.05 considered statistically significant. A univariate Cox proportional hazard model was employed to determine the effect of time on each molecule studied.

Data Analysis

The aims of this analysis are to 1) compare F actin, G actin, and F/G actin ratio over three time points (enrollment, 72 hours, and 7 days); 2) compare F and G actin between cases and controls; and 3) identify the effect of F/G actin ratio on the risk of death over time. Because many values were below the limit of detection (LOD), the standard practice of using half the LOD was used for these values (LOD-0.625, replacement-0.3125). There were three values above the LOD, and since their upper bound is theoretically infinite, they remained at the upper bound (40 μg/mL for cases' G actin, 10 μg/mL for controls' G actin) and were considered censored. Univariate Cox proportional hazards models were used to determine the effect of time point on F, G, and F/G actin, and stratified Cox proportional hazards models were used to compare F, G, and F/G actin between cases and controls, and to determine the hazard of death given F/G ratio. Statistical significance is set at p<0.05. All analyses were done using SAS 9.4, (SAS Institute Inc., Cary, N.C., USA).

Results:

The median G-actin levels on day zero were significantly increased in septic shock patients 24.60 μg/mL (IQR 21.61, 28.67) compared with healthy controls 4.46 μg/mL (IQR 3.62, 5.25), p<0.001. F-actin levels were also elevated in patients with septic shock 3.5 ng/mL (IQR 1.17, 7.12) versus controls (all values below lowest detection range of assay 0.62 ng/mL), p<0.001. TB4 levels were undetectable below the lowest detection range of the assay (<78 ng/mL) at all three time points in the septic shock group and medians were statistically lower than the median levels of TB4 in healthy controls, 121 ng/mL (IQR 39.0, 246.79), p<0.001. No statistical differences were observed when comparing G- and F-actin over time in those with septic shock, p>0.05.

Table 1 gives the descriptive statistics for all variables among cases. IQR is the difference between the first and third quartile.

TABLE 1
Descriptive statistics of cases (N = 26)

| | Median (Min, Max) or | |
|---|---|---|
| Variable | N (%) | IQR (25^{th}, 75^{th}) |
| F actin ENR | 3.49 (0.31, 28.65) | 5.41 (1.17, 7.12) |
| F actin 72 h | 4.19 (0.31, 23.68) | 3.08 (2.41, 5.49) |
| F actin 7 d | 2.56 (0.31, 20.01) | 3.33 (0.87, 4.20) |
| F control | 0.31 (0.31, 0.31) | 0 (0.31, 0.31) |
| G actin ENR | 24.60 (14.22, 40.0) | 7.06 (21.61, 28.67) |
| G actin 72 h | 24.48 (14.55, 40.0) | 7.04 (20.59, 27.63) |
| G actin 7 d | 25.12 (15.93, 36.61) | 12.09 (20.48, 32.57) |
| G control | 4.46 (2.70, 10.0) | 1.63 (3.62, 5.25) |
| F/G actin ENR | 15.56 (1.21, 99.93) | 16.5 (7.47, 24.0) |
| F/G actin 72 h | 15.24 (1.27, 59.20) | |
| F/G actin 7 d | 9.45 (0.88, 61.52) | |
| F/G actin control | 7.0 (3.13, 11.58) | 2.89 (5.85, 8.74) |
| TB4 ENR | 39 (39, 39) | |
| TB4 Control | 121.0 (39.0, 392.22) | 207.79 (39.0, 246.79) |

Table 2 gives the results of three separate univariate Cox proportional-hazards models that examine the effect of time on F, G, and F/G. There are no statistically significant changes between 0, 72, and 168 hours.

TABLE 2
F, G, and F/G over time (cases only)

| Independent | | |
|---|---|---|
| variable | HR (95% CI) | P-Value |
| F Actin | 1.021 (0.981, 1.062) | 0.3047 |
| G Actin | 0.984 (0.948, 1.020) | 0.3731 |
| F/G Ratio | 1.006 (0.994, 1.018) | 0.3391 |

Table 3 gives the results of three separate univariate Cox proportional-hazards models that compare F, G, and F/G between cases and controls. There are no statistically significant differences.

TABLE 3
F, G, and F/G case versus control

| Independent | | |
|---|---|---|
| variable | HR (95% CI) | P-Value |
| F Actin | 1.021 (0.981, 1.062) | 0.3047 |
| G Actin | 0.984 (0.949, 1.020) | 0.7817 |
| F/G Ratio | 1.006 (0.994, 1.018) | 0.9138 |

Table 4 gives the results of the univariate Cox proportional-hazards model examining the effect of F/G ratio, F clearance, F, G, and F/G ratio on the hazard of death (as a function of time). There is not a statistically significant effect of any of the measurements on the hazard of death.

TABLE 4
F/G over time, alive vs. dead

| Independent | | |
|---|---|---|
| variable | HR (95% CI) | P-Value |
| F/G Ratio | 1.008 (0.989, 1.027) | 0.6551 |
| F Clearance | 1.001 (0.997, 1.004) | 0.7517 |
| F (Enr) | 1.018 (0.935, 1.108) | 0.6808 |
| G (Enr) | 1.038 (0.982, 1.098) | 0.1867 |
| F/G Ratio (Enr) | 0.999 (0.972, 1.026) | 0.9418 |

Table 5 compares enrollment F actin to control F actin, and enrolment G actin to control G actin. It gives the median, minimum, and maximum values. Using a Wilcoxon two-group test means that the censoring cannot be taken into account, and so the values above the LOD remain at the LOD (40 and 10). All values of F actin for the controls and TB4 for the cases are below the LOD, so there is no variability to model and these results should be considered unreliable.

TABLE 5
Wilcoxon two-group tests

| | Controls | Cases (ENR) | P-Value |
|---|---|---|---|
| F actin | 0.31 (0.31, 0.31) | 3.49 (0.31, 28.65) | <0.001 |
| G actin | 4.46 (2.70, 10.0) | 24.60 (14.22, 40.0) | <0.001 |
| F/G actin | 7.0 | 15.56 | 0.0065 |
| TB4 | 121.0 (39.0, 392.22) | 39 (39, 39) | <0.001 |

Results:

26 septic patients with plasma samples available for this study were randomly selected from insert name of previous study/database. The mean age was 63 years of age and 69% were men. The average APACHEII score on patient arrival was 19.9 and overall hospital mortality rate was 38%. The average hospital length of stay was 34 days and average intensive care unit length of stay was 28 days. The 17 healthy controls consisted of a convenience sample of hospital employees who did not self-report illness during sample acquisition. Average age was 32 years of age and 47% were male. Descriptive statistics of G-actin, F-actin, and TB4 are presented in Table 1. Table 2 displays the results of the Wilcoxon two-group tests comparing levels of molecule in the septic shock group at day zero versus controls. Table 3 compares the levels of G-actin and F-actin in the septic shock group, and compares them over day zero, three, and seven.

G-Actin:

All serum samples in both the sepsis and control groups contained G-actin. The median level of the sepsis group at enrollment was 24.6 μg/mL (21.16, 28.67) which was greater than the healthy control group 4.46 μg/mL (3.62, 5.25), p<0.001. The medians of G-actin did not vary with time; T0 24.6 (21.16, 28.67) μg/mL, TD3 24.48 (20.59, 27.63) g/mL, and TD7 25.12 (20.48, 32.57) g/mL, p=0.37.

F-Actin:

The median concentrations of F-actin over time in the sepsis group did not vary over time; T0 3.49 (1.17, 7.12) ng/mL, TD3 4.19 (2.41, 5.49) ng/mL, and TD7 2.56 (0.87, 4.20) ng/mL, p=0.30. None of the 17 healthy control samples contained F-actin (lowest detection of ELISA Assay 0.625 ng/mL).

Thymosin Beta-4:

The median concentration for the healthy control group was 121 ng/mL (39.0, 246.79). No serum samples in the septic group contained detectable TB4 levels (lowest detection of ELISA Assay was 78 ng/mL). The levels between each group were statistically different (p<0.001).

Conclusions:

Septic shock is associated with increased levels of G-actin and F-actin as well as decreased levels of TB4 when compared to healthy controls. G-actin, but not F-actin, is present in healthy controls suggesting that serum F-actin may play a significant pathophysiologic role in septic shock, perhaps involved in the causative pathway of microcirculatory dysfunction. TB4 is likely decreased in patients with septic shock due to a consumptive process when it binds free G-actin in the serum, allowing for uncontrolled polymerization of F-actin.

This study is the first to quantify levels of G-actin and F-actin and correlate ratios of F-actin and G-actin in the assessment and prognosis in patients with septic shock. The data proposes that the levels of both molecules are significantly greater than those of healthy controls. The data also reveals that F-actin does not circulate in the serum of healthy controls (or at least is less than the lowest detection of the ELISA Assay of 0.625 ng/mL). This study is believed to be the first to quantify the levels of G-actin circulating in a normal population. Without being bound to any particular theory, the inventors hypothesize that in healthy patients, TB4 sequesters roaming G-actin in the serum, disfavoring the polymerization of F-actin and along with other Actin Binding Proteins (ABPs), eliminate F-actin from forming. The inventors postulate that the polymerized form of actin (F-actin) is deleterious to survival and partially responsible for the derangements seen in septic shock.

The data shows that TB4 is decreased in patients with septic shock. This is consistent with previous studies. When healthy controls were given a non-lethal dosage of the endotoxin lipopolysaccharide (LPS), levels of TB4 rapidly declined in the serum. Additionally, mice that were treated with TB4 and then exposed to an LD₅₀ dosage of LPS lived longer and had decreased levels of pro-inflammatory cytokines versus those who were not treated with TB4.

Overall, the excessive levels of F-actin in the serum of patients with sepsis and septic shock appear to be deleterious to survival. The data presented herein supports the development of clinical trials on the use of TB4 and other select ABPs as potential therapy for patients in septic shock. In addition, F-actin and G-actin, and ratios thereof, are potential novel biomarkers for sepsis, severe sepsis, septic shock and prognosis of septic shock.

The healthy controls consisted of a convenience sample of hospital employees and were not aged matched. They likely did not have equivalent co-morbidities as the sepsis group. Additionally, this was a retrospective study and samples were randomly picked from a database. The power of this study was very low between septic shock patients who lived and died (16 versus 10 respectively). This may explain the lack of differences between the levels of G-actin and F-actin amongst those patients who lived versus those who died. Experiments performed only enrolled patients who survived at least seven days in order to observe the trend over time. It is possible that the values of both forms of actin would have been greater earlier had we included patients who died before seven days. Finally, the study was limited in comparing TB4 over time as all values in our septic group were below the LOD.

### Example 2. Determining F-Actin and G-Act in n Healthy, Cardiac Surgery (No-Infections SIRS) and Septic Shock Subjects

Methods:

This study was performed in accordance with the ethical guidelines of Henry Ford Hospital and the study was approved by the Institutional Review Board (IRB #8485). Informed consent was obtained from healthy controls and the non-infectious SIRS group but was waived for those patients in septic shock. Plasma samples for the septic shock group were randomly selected from a previous cohort of stored samples. These patients were enrolled after they were identified as vasopressor dependent shock despite adequate fluid resuscitation and blood was collected at days 0, 3 and 7. Healthy controls consisted of a convenience sample of hospital employees who did not self-report illness during sample acquisition. The non-infectious SIRS group consisted of patients after they underwent a coronary artery bypass graft (CABG). Whole blood samples were obtained from each subject and collected into an EDTA-containing tube. Within sixty minutes of collection, samples were centrifuged at 2000 RPM for ten minutes and plasma was harvested. Specimens were then stored at −80° C. until analysis. Enzyme-linked immunosorbent assays (ELISA) were used to measure TB4 (Wuxi Donglin Sci&Tech Development Co, Jiangsu, China), F-actin (MyBiosource, San Diego, Calif.), and G-actin (MyBiosource). Samples were assayed separately for each analyte following the manufacturer's instructions. Sample concentrations were derived from plotting ODs of standards to create a standard curve (TB4) or from a four parameter logistic curve (4-PL) (F-actin and G-actin), using Graph Pad Prism 6 (GraphPad Software La Jolla, Cam USA). The reporting ranges for the different analytes were TB4 (78 ng/mL-10,000 ng/mL), F-actin (0.62 ng/mL-40 ng/mL), and G-actin (0.25 ng/mL-10 ng/mL). Due to previous data not published, the G-actin assay for the septic shock group was diluted 1:4 and the assay for the CABG patients was diluted 1:8 secondary to elevated levels compared to the assay range. All analytes were measured in duplicate. A Wilcoxon two-group test was used when comparing two medians against each other and a Kruskal-Wallis one-way analysis of variance by ranks was employed when comparing three groups together using SAS 9.4 (SAS Institute Inc., Cary, N.C., USA).

Results are reported in median (IQR) with a P value under 0.05 considered statistically significant. When an analyte fell below the lowest concentration of the assay, the value is reported as half of the lowest concentration (per industry standard). Those values exceeding the highest concentration of the assay were reported as the assays maximum value (per industry standard). Receiving Operator Characteristic (ROC) curves were created using GraphPad Prism. A univariate Cox proportional hazard model was employed to determine the effect of time on each analyte studied.

Results:

Patient characteristics of the septic shock, CABG, and healthy control groups are presented in Table 6.

TABLE 6
Characteristics of septic shock, CABG, and healthy control groups.

| | | | Healthy |
|---|---|---|---|
| | Septic Shock | CABG | Controls |
| | (N = 26) | (N = 10) | (N = 17) |
| Age - yr | 62.7 ± 16.1 | 64.6 ± 5.8 | 32.3 ± 7.2 |
| Male Sex - no. (%) | 18 (69) | 8 (80) | 8 (47) |
| APACHE 2 Score enr¶ | 19.9 ± 9.8 | 7.7 ± 1.8 | NA |
| Hospital Length of Stay - days | 34.5 ± 24.9 | 14 ± 5.1 | NA |
| ICU Length of Stay - days | 28.2 ± 27.0 | 4.4 ± 1.2 | NA |
| Hospital Mortality - no. (%) | 10 (38) | 0 (0) | NA |

Plus-minus values are means ± SD.
¶information on APACHE 2 score on enrollment was missing for one patient in the septic shock group.
yr = years,
no = number,
enr = enrollment,
NA = not applicable.

Septic Shock: Twenty-six patients were enrolled in the vasopressor dependent septic shock group. The mean age was 62.7 years and 69% were men. The mean APACHE II score on patient time of vasopressor dependent septic shock was 19.9 and the overall hospital mortality rate was 38%. The mean hospital length of stay was 34.5 days and average intensive care unit length of stay was 28.2 days. Most patients (25/26) were enrolled in the surgical intensive care unit and one patient was enrolled in the medical intensive care unit. The most common site of infection was the abdomen, followed by lung and soft tissues with two patients having more than one site of infection. Various cultures were available resulting in culture positivity in 23/26 (88.5%) patients (Table 7).

TABLE 7
Characteristics of septic shock at enrollment.

| | |
|---|---|
| Culture positive any site | 23/26 (88.5%) |
| Blood positive culture | 5/25 (20.0%) |
| Urine culture positive | 6/22 (27.3%) |
| BAL culture positive | 15/20 (75.0%) |
| Fungal blood culture positive | 0/17 (0.0%) |
| MRSA nasal swab positive | 2/24 (8.3%) |
| Wound culture positive | 11/15 (73.3%) |
| C. diff stool positive | 7/17 (41.2%) |
| Site of Infection | |
| Abdomen | 18 |
| Pulmonary | 7 |
| Soft Tissue | 4 |
| Urogenital | 0 |
| Respiratory | |
| No ARDS | 9/26 (34.6%) |
| Signs of ARDS | 17/26 (65.4%) |
| Mild ARDS PaO₂/FiO₂ 201-300 | 8/17 (47.0%) |
| Moderate ARDS PaO₂/FiO₂ 101-200 | 9/17 (52.9%) |
| Severe ARDS PaO₂/FiO₂ <100 | 0/17 (0.0%) |
| Liver | |
| Bilirubin <1.2 mg/dL (No ALF) | 3/10 (30.0%) |
| Bilirubin ≧1.2 mg/dL (ALF) | 7/10 (70.0%) |
| Albumin <3.4 mg/dL (No ALF) | 0/10 (0.0%) |
| Albumin <2.5 mg/dL (ALF) | 3/10 (30.0%) |
| Albumin <2.0 mg/dL (ALF) | 7/10 (70.0%) |
| INR >1.5 (ALF) | 10/19 (52.6%) |
| Kidney | |
| Acute kidney injury | 11/21 (52.4%) |
| No acute kidney injury | 10/21 (47.6%) |
| Chronic dialysis | 3 |
| Urine output not recorded | 2 |

BAL = Bronchoalveolar lavage,
MRSA = Methicillin-resistant Staphylococcus aureus,
C. diff = Clostridium difficile,
ARDS = Acute Respiratory Distress Syndrome,
ALF = Acute Liver Failure,
INR = International Normalized Ratio.
Some patients had multiple sites of infection.

Acute kidney injury was defined by urine output of less than 0.5 mL/kg of body weight within 6 hours of onset of shock and was present in 11/21 (52.4%) patients (data not available on 2 patients, 3 patients on chronic dialysis). Acute respiratory distress syndrome with PaO₂/FiO₂ levels below 300 at the time of enrollment was present in 17/26 (65.4%). Acute liver failure with bilirubin elevations of greater than or equal to 1.2 mg/dL was present in 7/10 (70%) at the time of enrollment. International normalized ratio levels were greater than 1.5 in 10/19 (52.6%) patient at time of enrolment. Albumin levels are known to be diminished in patients in the critical care setting. Patients following fluid resuscitation also may have temporarily decreased levels of albumin not necessarily representing acute liver injury. Ten out often (100%) patients had albumin levels below 2.5 at the time of enrollment.

CABG: A total of ten patients were enrolled in the non-infectious SIRS group. The average age was 64.6 years, 80% were male, and the average APACHE II score was 7.7. None of the CABG patients died during their hospital stay. The median time from completion of the CABG surgery to blood sample collection was 28 hours. Eight out of the ten CABG patients had two or more SIRS criteria present at time of blood collection and all ten had at least one SIRS criteria. One out of ten patients were off pump for their procedure whereas nine patients required cardiopulmonary bypass for an average of 139 minutes.

Healthy Controls: Seventeen healthy individual blood samples were obtained for the healthy control group. Mean age was 32.3 years and 47% were male.

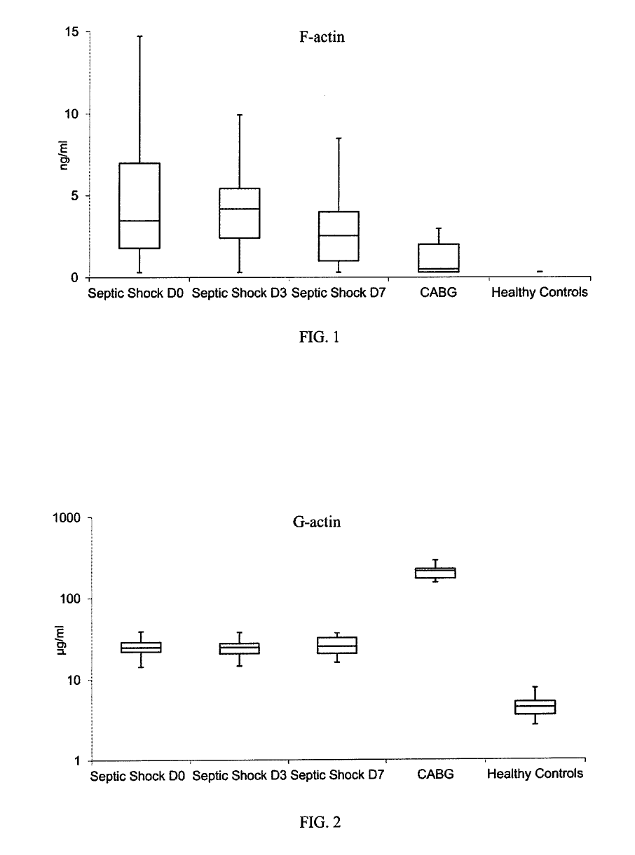

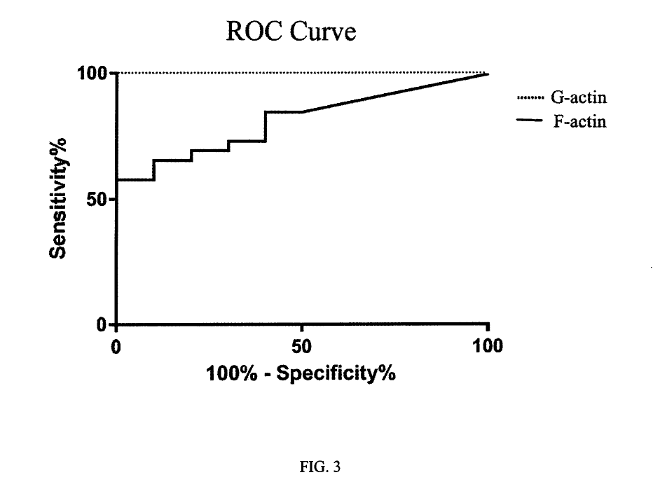

F-Actin: For graphical description please refer to FIG. 1. 22/26 (84.6%) patients in the septic shock group (at time of enrollment) and 5/10 (50%) patients in the CABG group had detectable plasma levels of F-actin above the lowest detection of the ELISA Assay (0.62 ng/mL). No healthy control patients had detectable levels of F-actin above the minimum detection range of the assay. The median concentration of F-actin in the septic shock group was 3.49 ng/mL (1.62, 7.18) and in the CABG group was 0.51 (0.31, 2.13). The ROC curve for F-actin, septic shock at time of enrollment versus CABG is displayed in FIG. 3. The area under the curve (AUC) is 0.812 (95% CI 0.67-0.95). At a cut off value of 3.02 ng/mL the sensitivity is 57.7% (95% CI 36.92-76.65) and specificity is 100% (95% CI 69.15-100). The median concentrations of F-actin over time in the septic shock group did not vary; day zero 3.49 ng/mL (1.17, 7.12), day three 4.19 ng/mL (2.41, 5.49), and day seven 2.56 ng/mL (0.87, 4.20), p=0.30.

G-Actin: For graphical description please refer to FIG. 2. All samples in each group contained measurable G-actin levels in plasma. The median level of G-actin in the non-infectious SIRS group was 214.4 μg/mL (167.7, 241.31), in the septic shock group was 24.6 μg/mL (21.61, 28.67) and in the healthy control group was 4.46 μg/mL (3.62, 5.25), p<0.0001. The ROC curve for G-actin, septic shock at time of enrollment versus CABG at time of enrollment is displayed in FIG. 3. The AUC is 1.0 (95% CI 1.0-1.0). At a cut off value of 97.49 μg/mL the sensitivity is 100% (95% CI 86.77-100) and specificity is 100% (95% CI 69.15-100%). The median levels of G-actin did not vary with time amongst the septic shock group; day zero 24.6 μg/mL (21.61, 28.67), day three 24.48 μg/mL (20.59, 27.63), and day seven 25.12 μg/mL (20.48, 32.57), p=0.37.

Thymosin Beta-4: None of the samples in the septic shock and non-infectious SIRS contained detectable TB4 levels (lowest detection of ELISA Assay was 78 ng/mL) while 12/17 of the healthy control group detected a signal with our ELISA Assay. The median concentration for the healthy control group was 121 ng/mL (39.0, 246.79). The levels between all three groups were statistically different (p<0.0001). The ROC curve for TB4, septic shock versus healthy control at time of enrollment had a specificity of 71% and sensitivity of 100% at a cutoff value of 68.9 ng/mL. The AUC was 0.85 (95% CI 0.72-0.99). None of the values on day three or seven in the septic shock group contained values within our assay range. The F/G Actin Ratio: The median F/G actin ratio in the healthy control group was 0.0015 (0.0007, 0.0026) versus in the septic shock group was 0.007 (0.00058, 0.00087), p<0.05.

Discussion

The polymerization of the monomeric G-actin into its filamentous F-actin is tightly regulated by ABPs such as TB4. Our investigational study is the first believed to quantify the plasma levels of G-actin and F-actin in humans. The experimental data provides that both G-actin and F-actin were significantly elevated in patients with septic shock when compared to healthy controls likely secondary to actin release during sepsis-associated cellular death. This is consistent with a previous qualitative study showing circulating actin in sepsis (Lee 2008). Our results differ from Lee et al in that their study found no detectable levels of actin in healthy controls but our results show detectable levels of G-actin. It is possible that the anti-actin antibodies used in the 2008 study were not sensitive to the globular form of actin and thus concluded that actin was not present in normal controls. Besides being a marker of disease, it is possible that F-actin may play a role in microcirculatory dysfunction in septic shock by creating long filamentous chains disturbing microcirculatory flow. A study in which rats were exposed to increasing amounts of globular actin intravenously resulted in filament formation, microthrombi, and endothelial injury, all present in sepsis. These changes were not observed when actin was preincubated with an actin binding protein (Haddad 1990). In addition, exogenous infusion of actin binding proteins that regulate the conversion of G- to F-actin have decreased mortality in two septic animal models (Badamchian 2003, Lee 2007). Perhaps, the inflammatory insult occurring during a CABG does not overwhelm ABP's ability to neutralize the conversion of G-actin to F-actin resulting in a greater ratio of F:G-actin in patients with septic shock.

Levels of TB4 were also studied, and the experimental data found no detectable levels above the lowest detection range in the assays used in the septic shock or non-infectious SIRS group. However, many of the healthy controls had levels above the lowest detection range. The experimental findings are consistent with previous work by Badamchian et al in which a non-lethal dosage of the endotoxin lipopolysaccharide (LPS) was injected into healthy controls resulting in a rapid decline in levels of TB4. Additionally, pretreatment of TB4 in rats exposed to an LD₅₀ dosage of LPS lived longer and had decreased levels of pro-inflammatory cytokines versus those who were not treated with TB4 (Badamchian 2003). It is possible that the release of actin into circulation results in consumption of TB4 as it binds free actin.

Conclusions: Septic shock is associated with significantly elevated levels of G-actin and F-actin as well as decreased levels of TB4 as compared to healthy controls. The levels of F-actin were greatest in patients with septic shock as compared to non-infectious SIRS and healthy controls with an AUC of 0.812 (septic shock versus non-infectious SIRS) suggesting a role as a biomarker in the diagnosis and treatment of septic shock.

What is claimed is:

1. A method for treating or preventing septic shock in a non-infectious or infectious SIRS subject, the method comprising:
(a) obtaining a blood sample from the non-infectious SIRS subject or the infectious SIRS subject;
(b) determining the amount of F-actin in the non-infectious SIRS subject or the infectious SIRS subject's blood sample;
(c) determining that the non-infectious SIRS subject or the infectious SIRS subject is in septic shock if the non-infectious SIRS subject or the infectious SIRS subject's F-actin level is about 3 ng/mL or greater; and
(d) administering an effective treatment to treat or prevent septic shock in the non-infectious SIRS subject or the infectious SIRS subject having an F-actin level of about 3 ng/mL or greater.

2. The method of claim 1, wherein the infectious SIRS subject presents with at least two SIRS criteria at the time of assessment.

3. The method of claim 1, wherein the non-infectious SIRS subject presents with zero or one SIRS criteria at the time of assessment.

4. The method of claim 2, wherein the at least one SIRS criteria comprises: elevated heart rate, elevated respiratory rate, an elevated or decreased temperature from 37° C., or an elevated or decreased white blood count.

5. The method of claim 1 wherein the blood sample is a plasma sample.

6. The method of claim 1, wherein determining the amount of F-actin in the non-infectious SIRS subject or the infectious SIRS subject's blood sample comprises assaying the amount of F-actin protein in the blood sample.

7. The method of claim 6, wherein determining the amount of F-actin protein in the non-infectious SIRS subject or the infectious SIRS subject's blood sample comprises measuring the amount of F-actin bound to an antibody which binds to F-actin and comparing the amount of bound antibody to a standard curve in an immunoassay.

8. The method of claim 7, wherein determining the amount of F-actin protein in the non-infectious SIRS subject or the infectious SIRS subject's blood sample comprises measuring the amount of F-actin in an ELISA assay.

9. The method of claim 1, wherein administering an effective treatment to treat or prevent septic shock in the non-infectious SIRS subject or the infectious SIRS subject comprises treating the non-infectious SIRS subject or the infectious SIRS subject with plasmapheresis, high dose ultrafiltration, extracorporeal membrane oxygenation, selective cytopheresis, selective antigen removal, continuous renal replacement therapy, or combinations thereof.

10. The method of claim 1, wherein the non-infectious SIRS subject or the infectious SIRS subject having a blood F-actin level of less than about 3 ng/mL is treated with anti-inflammatories, antibiotics, or combinations thereof.

11. A method for the detection of sepsis or septic shock in a human subject confirmed with SIRS, the method comprising: (a) quantifying a level of F-actin in a blood sample of said human obtained on days 0, 1 or 2 after subject is confirmed with SIRS, (b) determining whether the level of F-actin quantified in said serum sample is above about 3 ng/mL, and (c) predicting that the human will develop sepsis or septic shock when the level of F-actin quantified in said blood sample is above about 3 ng/mL.

12. The method of claim 11 wherein the level of F-actin is determined by ELISA, RIA, EIA, mass spectrometry, or microarray analysis.

13. The method of claim 11 wherein the level of F-actin is determined by a sandwich ELISA, wherein microtiter plates are coated with one type of antibody directed against F-actin, the plates are then blocked and the sample or a standard is loaded, a second type of antibody against F-actin is applied, a third antibody detecting the second antibody conjugated with a suitable label is then added, and the label used to quantify the level of F-actin.

14. The method of claim 13 wherein the label in the sandwich ELISA is an enzyme for chromogenic detection.

15. A method for in vitro establishing a prognosis for a SIRS subject of developing septic shock, consisting of the following steps: (i) obtaining a plasma sample from the subject, measuring the level of F-actin in the sample, by immunoassay; (ii) comparing the level of F-actin to a predetermined threshold plasma level of F-actin indicative for developing septic shock, wherein: if the level of F-actin in the plasma sample is above the predetermined threshold, the prognosis is that the SIRS subject will develop severe sepsis or septic shock; and if the level of F-actin in the plasma sample is below the predetermined threshold, the prognosis is that the subject will not develop septic shock

16. The method of claim 15, wherein said plasma sample has been collected at day 0, day 1 or day 2 after the onset of SIRS.

17. The method of claim 15, wherein said immunoassay is performed with an antibody which specifically binds to the F-actin.

18. The method of claim 17, wherein said antibody is fluorescently labeled.

19. The method of claim 15, wherein said immunoassay is an enzyme-linked immunosorbent assay (ELISA).

20. The method of claim 15, wherein the predetermined threshold for the prognosis of developing septic shock is an F-actin level in the plasma of about 3 ng/mL or greater.
